(19) United States
(12) Patent Application Publication
(10) Pub. No.: US 2017/0291958 A1
(43) Pub. Date: Oct. 12, 2017

(54) Compositions and Methods For the Modulation of Ras Proteins

(71) Applicant: NEW YORK UNIVERSITY, New York, NY (US)

(72) Inventors: Wei Dai, Saddle River, NJ (US); Mark R. Philips, New York, NY (US); Byeong Hyeok Choi, Hackensack, NJ (US)

(21) Appl. No.: 15/485,038

(22) Filed: Apr. 11, 2017

Related U.S. Application Data
(60) Provisional application No. 62/320,949, filed on Apr. 11, 2016.

(51) Int. Cl. C07K 16/32; C12N 9/14; A61K 31/352

(52) U.S. Cl. CPC C07K 16/32; A61K 31/352; C12N 9/14; C12Y 306/05002; C07K 2317/76; C07K 2317/34

(57) ABSTRACT

Described herein is a method of modulating sumoylation of a Ras protein by small ubiquitin-like modifier (SUMO) proteins. Provided herein is a method for regulating the activity of a Ras protein. Also provided is a treatment of proliferative diseases, such as cancer, by introducing specific mutations to a mutant Ras protein that is associated with the proliferative disease. Described herein is a modified Ras protein. Described herein are recombinant vectors, cells comprising the vectors expressing the modified Ras proteins. Provided herein is an antibody that specifically binds to a sumoylated Ras protein. Described herein is a method for identifying an agent that interferes with the sumoylation of a Ras protein. Also described herein are therapeutic and prophylactic compositions. Also provided herein is a method of using a modified Ras protein to replace an endogenous mutant Ras protein that is associated with a proliferative disease.

### STATEMENT OF GOVERNMENT SUPPORT

This invention was made with government support under US Public Service Awards CA090658, CA150512, CA100498, CA 116034, CA153354 and NIEHS center grant (ES000260). The government may have certain rights in this invention.

### RELATED APPLICATIONS

This application claims priority to Provisional Application Ser. No. 62/320,949, filed Apr. 11, 2016 which is incorporated by reference in its entirety.

### INCORPORATION-BY-REFERENCE OF SEQUENCE LISTING

A sequence listing, created on April 11, 2017 as the ASCII text file “10039-004622-US1_Seq_Listing.txt” having a file size of 14 kilobytes, is incorporated herein by reference in its entirety.

### 1. INTRODUCTION

The present disclosure provides compositions and methods for modulating the activities of the Ras proteins through modifying sumoylation of the Ras proteins by small ubiquitin-like modifier (SUMO) proteins for the treatment of diseases.

### 2. BACKGROUND

RAS proteins are among the mostly studied proto-oncogene products because of their frequent mutations in human malignancies. HRAS, KRAS and NRAS are three related proteins that are commonly expressed in mammalian cells and have overlapping but distinctive functions (Ref. 1-4). RAS proteins are GTPases that participate in multiple signaling cascades, regulating crucial cellular processes including cell survival, proliferation, differentiation, and autophagy (Ref. 2, 5, 6). RAS proteins are membrane-anchored proteins whose activity, subcellular localization, and stability are tightly controlled as deregulated expression and/or its activities frequently lead to malignant transformation (Ref. 1).

RAS proteins are regulated by post-translational modifications include farnesylation, carboxylmethylation, and palmitoylation (Ref. 1, 7, 8, 9, 10). Nascent RAS proteins are first processed by three-step modifications that lead to generation of lipidated proteins with hydrophobic C-termini that mediate association with cellular membranes (Ref 1). RAS proteins are also modified by palmitoylation through covalent attachment of 16-carbon palmitoyl chain to cysteine residues. Palmitoylation is a necessary step for translocation of RAS proteins from the endomembrane system to the cell surface membrane (Ref 1, 7).

Extensive research in the past has also revealed that RAS proteins are subjected to other posttranslational modifications including phosphorylation, ubiquitination, acetylation, and S-nitrosylation (Ref 8, 11, 12, 13, 14, 15, 29). For example, KRAS4B is phosphorylated on serine 181 by protein kinase C and the phosphorylation is involved in the negative regulation of its association with the plasma membrane. RAS proteins are also modified by monoubiquitination and bi-ubiquitination (Ref 11, 12; 13, 29). Lys117, Lys147 and Lys170 are likely the sites of ubiquitination 1. The E3 ligase specific for RAS ubiquitination is RABEX5 (Ref 13, 29). Cys118, a highly conserved site in RAS isoforms and orthologues, can be modified by nitrosylation (Ref 16). S-nitrosylation facilitates guanine nucleotide exchange, promoting efficient RAS activation (Ref 16, 17).

The SUMO (small ubiquitin-related modifier) pathway resembles the ubiquitin pathway. It consists of three enzymatic components including the E1 activating enzyme (SAE1/2), the E2 conjugating enzyme (UBC9) and a series of E3 ligases that promote sumoylation in a substrate-specific manner (Ref 18, 19). Sumoylation occurs on lysine residues of target proteins immediately after amino acids of a hydrophobic nature. Mammalian cells have three structural homologs: SUMO1, SUMO2, and SUMO3. Similar to other types of posttranslational modifications, sumoylation is reversed by the proteolytic cleavage of isopeptidases of the SENP family (Ref 19). Extensive research has uncovered important functions of sumoylation that control the subcellular localization, stability and enzyme activities of target proteins. Sumoylation is also critical in development and cell biology, as its disruption either causes abnormal cellular growth and differentiation or leads to embryonic lethality (Ref. 20).

Described herein is that sumoylation plays an important role in regulating the stability, activity, and subcellular localization of the Ras proteins. HRAS. KRAS and NRAS are all SUMO-modified. In certain embodiments, SUMO3 is the modifier for a Ras protein. In certain embodiments, Lys42 is the primary residue for sumoylation of a Ras protein. In certain embodiments, PIASγ is an E3 ligase promoting RAS sumoylation. In certain embodiments, sumoylation is essential for activating RAS and its downstream signaling. In certain embodiments, a SUMO-resistant mutant of a Ras protein (e.g., KRAS) has reduced cellular migration and invasiveness in vitro and has significantly suppressed tumor formation in vivo.

Cancer is one of the most deadly diseases in the present world. Facing cancer, most people believe surgery, chemotherapy or radiation therapy is the only possible solution. However, not all cancer patients are suitable for surgery, and cancer metastasis may cause the failure of surgery treatment. The chemotherapy or radiation therapy may lead to large damage of normal organs and less effect on cancer niche. Thus, there is a need for a new method of treating cancer and other proliferative diseases.

### 3. SUMMARY

The disclosure is based, in part, on the Applicant's discovery that RAS proteins are sumoylated by small ubiquitin-like modifier (SUMO) proteins. Blocking sumoylation of RAS with an antagonists, a small molecule, antibodies, peptides may inhibit cancer development.

Described herein is a method of modulating sumoylation of a RAS protein by a SUMO protein. Also provided is a method for modulating the activity of a RAS protein. In certain embodiments, the method comprises contacting the cell with an agent, where the agent interferes with sumoylation of the Ras protein at amino acid residue 42 by a small ubiquitin-like modifier (SUMO) protein.

In one embodiment, the RAS protein has a mutation that causes cancers. In one embodiment, the RAS protein is an oncogenic RAS protein. In certain embodiments, the activities of a RAS protein includes: (i) RAS activation; (ii) sustained activation; and (iii) downstream signaling. In one embodiment, the conjugation of a RAS protein to SUMO protein is inhibited by small molecules, peptides, antibodies or fragments thereof, and/or inhibitory nucleic acids. In one embodiment, the small molecule is a SUMO E2 inhibitor (2-D08). In one embodiment, the small molecule 2-D08 blocks K-RAS sumoylation. In one embodiment, the small molecule 2-D08 suppresses pancreatic cell migration. In one embodiment, the small molecule 2-D08 suppresses pancreatic cell migration in cells that comprises a K-RAS mutation.

Provided herein is a method for decreasing sumoylation of a RAS protein by a SUMO protein wherein the RAS protein comprises a first amino acid residue substituted at position 12, 13, 17, 35, 59, 60, 61, and/or 119. The method comprises: introducing a second amino acid residue substituted at position 42 of SEQ ID NO:2 to form a modified RAS protein. In one embodiment, the first amino acid residue: (i) at position 12 is substituted with valine or an amino acid other than glycine; (ii) at position 13 is substituted with an amino acid other than glycine; or (iii) at position 61 is substituted with an amino acid other than glutamine. In one embodiment, the second amino acid residue at position 42 is substituted with arginine, lysine or histadine. In one embodiment, the SUMO protein is SUMO-3. In one embodiment, the decreased sumoylation of the RAS protein decreases or inhibits cell proliferation and/or migration. In one embodiment, the modified RAS has reduced activities. In one embodiment, the downstream signaling of the modified RAS is reduced. In one embodiment, the decreased sumoylation of the RAS protein decreases downstream p-ERK levels.

In certain embodiments, the mutation of RAS protein is introduced through recombinant techniques that are known in the art. In one embodiment, the mutation of RAS protein is carried out through gene therapy in a subject. In certain embodiments, therapeutic and pharmaceutical compositions comprise vectors expressing nucleic acid molecules.

Provided herein is a method for regulating activity of a RAS protein, comprising: contacting the Ras protein with a recombinant molecule, wherein the recombinant molecule interferes with the conjugation of the Ras protein at position 42 of SEQ ID NO:2 to a SUMO protein. In one embodiment, the recombinant molecule is an antibody or a fragment thereof. In one embodiment, the antibody or a fragment thereof specifically binds to an epitope comprising amino acid residue 42 of SEQ ID NO: 2. In one embodiment, the SUMO protein is SUMO-3. In one embodiment, the activity of the RAS protein is reduced. In one embodiment, the downstream signaling of the RAS protein is reduced. In one embodiment, the downstream p-ERK levels are decreased. In one embodiment, cell proliferation is inhibited.

Provided herein is a treatment of a proliferative disease, such as cancer. More specifically, the disclosure relates to the treatment and prevention of cancers including, but not limited to, lung cancer, pancreatic cancer, colon, cancer, melanomas, hematopoietic tumors and thyroid cancer using compositions which can be used as therapeutic agents which are capable of selectively inhibiting the early events that are associated with cancer development. The present disclosure further relates to methods for using the therapeutic compositions.

Provided herein are methods relating to inhibiting or reducing proliferation of a cancer cell, for treating cancer in a subject in need of treatment. The method comprises decreasing conjugation of a SUMO protein to a RAS protein. In one embodiment, provided herein is a treatment of a proliferative disease, such as cancer, by introducing specific mutations in a RAS protein. In one embodiment, the mutation of RAS protein is carried out through gene therapy in a subject. In one embodiment, the mutation of RAS protein is carried out through cluster regularly interspaced short palindromic repeat-associated nuclease (“CRISPR”) technology well known in the art. In certain embodiments, the therapeutic and pharmaceutical compositions comprise vectors expressing DNA and/or RNA. In certain embodiments, the therapeutic and pharmaceutical compositions comprise a modified RAS or an agent that interferes with the conjugation of a RAS protein to a SUMO protein. In certain embodiments, the method of treatment of cancer is in combination with other cancer treatment. In certain embodiments, the cancer treatment is chemotherapy, radiation therapy, gene therapy, surgery or a combination thereof.

Provided herein is a modified RAS protein comprising: a first amino acid residue substituted: (i) at position 12 with a valine, or an amino acid other than glycine; (ii) at position 13 with an amino acid other than glycine; or (iii) at position 61 with an amino acid other than glutamine; and a second amino acid residue substituted at position 42 of SEQ ID NO:2 with an amino acid other than Lysine. In one embodiment, the second amino acid residue at position 42 is substituted with arginine.

Described herein are nucleic acid, recombinant vectors, recombinant cells comprising the vectors expressing the modified RAS protein.

Provided herein is an antibody or a fragment thereof that specifically binds to a modified RAS protein or to a sumoylated protein formed by the conjugation of a RAS protein to a SUMO protein. In one embodiment, the antibody or a fragment thereof recognizes an epitope comprising an amino acid at position 42 of SEQ ID NO:2. In one embodiment, the antibody or a fragment thereof specifically binds to a non-sumoylated Ras protein, but does not bind to a sumoylated Ras protein. Provided herein is an antibody or a fragment thereof that specifically binds a sumoylated RAS protein. In one embodiment, the antibody or a fragment thereof specifically binds to a sumoylated Ras protein, but does not bind to a non-sumoylated Ras protein.

Described herein is a method for identifying an agent that interferes with the conjugation of a SUMO protein to a RAS protein. In one embodiment, provided herein is a method for identifying an agent that interferes with the sumoylation of a RAS protein, said method comprises: (i) contacting a RAS protein with a labeled agent in the presence of the SUMO protein; (ii) detecting binding of the labeled agent with RAS or binding of the labeled agent with the SUMO protein; and (iii) isolating the labeled agent that binds to RAS or SUMO protein. In one embodiment, the SUMO protein is SUMO-3.

Also disclosed is a method for screening potential antagonists to RAS sumoylation comprising: (i) administering a test compound to a cell expressing RAS and SUMO; (b) measuring the level of sumoylated RAS; and (c) determining whether the test compound reduces the level of RAS sumoylation, wherein the test compound that results in decreased RAS sumoylation are identified as the antagonists. In one embodiment, the SUMO protein is SUMO-3.

In certain embodiments, the agent or the potential antagonist to RAS sumoylation is an antibody or a small molecule.

Also described herein are therapeutic and prophylactic compositions. In certain embodiments, the therapeutic and prophylactic compositions contain a purified form of the modified RAS protein or an agent/antagonist that interferes with the sumoylation of a RAS by a SUMO protein. Also provided is a method of administering an effective amount of the therapeutic and prophylactic compositions to a subject in need thereof.

In one aspect, the modified RAS protein replaces an endogenous RAS protein that carries a mutation that causes a proliferative disease. In certain embodiment, the modified RAS protein is expressed in a cancer, retarding its growth. In one aspect, the cancer is a solid tumor. In certain embodiments, the tumor is a malignant tumor. In certain embodiments, the cancers include, but are not limited to, lung cancer and pancreatic cancer. In certain embodiments, the cancers include colon cancer, melanomas, hematopoietic tumors and thyroid cancer.

Also described herein is a kit comprising the agents or compositions described herein and a pharmaceutically acceptable carrier.

Provided herein is a fusion protein comprising a RAS protein having a Flag-tag and a SUMO-3 protein having a human influenza hemagglutinin (HA)-flag.

Provided herein is a cell comprising a vector expressing a fusion protein comprising a RAS protein having a Flag-tag and a SUMO-3 protein having a human influenza hemagglutinin (HA)-flag.

Provided herein is a method for inhibiting sumoylation of RAS by a SUMO protein in a cell comprising introducing an antibody that specifically binds to PIASγ in the cell.

Provided herein is a method for inhibiting sumoylation of RAS by a SUMO protein in a cell comprising introducing an inhibitor of PIASγ in the cell.

Provided herein is a method for inhibiting sumoylation of RAS by a SUMO protein in a cell comprising introducing an activator of SENP1 and/or SENP2 in the cell.

Provided herein is a method for increasing sumoylation of RAS by a SUMO protein in a cell comprising introducing an antibody that specifically binds to SENP1 and/or SENP2 in the cell.

Provided herein is a method for diagnosing cancer in a subject using the antibody that specifically binds a sumoylated RAS. In certain embodiments, the cancer is lung cancer or pancreatic cancer.

The present disclosure provides for a method for modulating (e.g., reducing or enhancing) the activity of a Ras protein in a cell. In certain embodiments, the method comprises the step of contacting the cell with an agent that interferes with sumoylation of the Ras protein at amino acid residue 42 and/or amino acid residue 104 by a small ubiquitin-like modifier (SUMO) protein.

The present disclosure also provides a method for decreasing proliferation and/or migration of a cancer cell. In certain embodiments, the method comprises the step of contacting the cell with an agent that interferes with sumoylation of the Ras protein at amino acid residue 42 and/or amino acid residue 104 by a small ubiquitin-like modifier (SUMO) protein.

The present disclosure provides a method of treating cancer in a subject. In certain embodiments, the method comprises the step of administering to the subject an agent that interferes with sumoylation of the Ras protein at amino acid residue 42 and/or amino acid residue 104 by a small ubiquitin-like modifier (SUMO) protein.

The cancer cell may be a lung cancer cell or a pancreatic cancer cell. The cancer may be lung cancer, pancreatic cancer, or any cancer described herein.

Also encompassed by the present disclosure is a method for decreasing sumoylation of a Ras protein by a small ubiquitin-like modifier (SUMO) protein. In certain embodiments, the method comprises the step of introducing a first point mutation at amino acid residue 42 and/or amino acid residue 104 of the Ras protein to form a modified Ras protein. In one embodiment, the decreased sumoylation of the Ras protein decreases cell proliferation and/or migration. In one embodiment, the decreased sumoylation of the Ras protein decreases reduces activity of downstream Ras effector protein. For example, the decreased sumoylation of the Ras protein decreases the level of phosphorylated ERK (p-ERK). In certain embodiments, the Ras protein is a mutant Ras protein. In certain embodiments, the mutant Ras protein may comprise a second point mutation at a second amino acid residue, e.g., at amino acid residue 12, 13, 17, 35, 59, 60, 61 and/or 119 of the Ras protein. In certain embodiments, the mutant Ras protein is an oncogenic Ras protein. In certain embodiments, the mutant Ras protein is an activated Ras protein which refers to a mutated form of a Ras protein which is capable of inducing cells to lose their normal growth control. In certain embodiments, the mutant Ras protein comprises the amino acid sequence set forth in SEQ ID NO: 2 (or SEQ ID NO: 4, or SEQ ID NO: 6, or SEQ ID NO: 8) comprising the first point mutation at amino acid residue 42 and/or amino acid residue 104 of SEQ ID NO: 2 (or SEQ ID NO: 4, or SEQ ID NO: 6, or SEQ ID NO: 8). In certain embodiments, the mutant Ras protein comprises the amino acid sequence set forth in SEQ ID NO: 2 (or SEQ ID NO: 4, or SEQ ID NO: 6, or SEQ ID NO: 8) comprising the second point mutation. In certain embodiments, the second amino acid residue at amino acid residue 12 is substituted with valine or an amino acid other than glycine, at amino acid residue 13 substituted with an amino acid other than glycine, at amino acid residue 17 substituted with asparagine or an amino acid other than serine, at amino acid residue 61 substituted with an amino acid other than glutamine, and/or at amino acid residue 119 substituted with asparagine or an amino acid other than aspartic acid.

The present method may modulate (e.g., reduce or enhance) the activity of the Ras protein.

The present method may modulate (e.g., reduce or enhance) the activity of at least one downstream Ras effector as described herein. For example, the present method may decrease the level of phosphorylated ERK (p-ERK).

The present method may modulate (e.g., reduce or enhance) cell proliferation and/or migration.

The SUMO protein may be SUMO1, SUMO2, SUMO3 or SUMO4. In certain embodiments, the SUMO protein is SUMO3.

The agents for use in the present methods and compositions can be any of a small molecule, an antibody, an antibody fragment, a polypeptide, a carbohydrate, an inhibitory nucleic acid, or any combination thereof.

In certain embodiments, the agent is an antibody or a fragment thereof.

In certain embodiments, the antibody or a fragment thereof specifically binds to an epitope comprising amino acid residue 42 or amino acid residue 104 of the Ras protein. Amino acid residue 42 or amino acid residue 104 of the Ras protein may or may not be sumoylated by a SUMO protein. In certain embodiments, the antibody or a fragment thereof specifically binds to an epitope comprising amino acid residue 42 or amino acid residue 104 of the Ras protein, where the Ras protein is sumoylated at amino acid residue 42 or amino acid residue 104.

In certain embodiments, the agent is a SUMO E1 inhibitor, such as an inhibitor of SAE (e.g., SAE1 or SAE2). The agent may be a small molecule which inhibits a SUMO E1. The agent may be an antibody or an antibody fragment which specifically binds to a SUMO E 1. The agent may be an inhibitory nucleic acid as described herein which specifically binds to a nucleic acid encoding a SUMO E1.

In certain embodiments, the agent is a SUMO E2 inhibitor, such as an inhibitor of UBC9. The agent may be a small molecule which inhibits a SUMO E2. The agent may be an antibody or an antibody fragment which specifically binds to a SUMO E2. The agent may be an inhibitory nucleic acid as described herein which specifically binds to a nucleic acid encoding a SUMO E2. Non-limiting examples of SUMO E2 inhibitors include 2-(2,3,4-Trihydroxyphenyl)-4H-chromen-4-one (2-D08, also named 2-(2,3,4-Trihydroxyphenyl)-4H-1-Benzopyran-4-one or 2′,3′,4′-trihydroxy-flavone). In some embodiments, the SUMO E2 inhibitor is 2-(2,3,4-Trihydroxyphenyl)-4H-chromen-4-one (2-D08) including derivatives, analogs, chemical analogs, salts, variants, and modifications thereof.

In certain embodiments, the agent is a SUMO E3 inhibitor, such as an inhibitor of a PIAS protein (e.g., PIAS1, PIAS3, PIAS2 such as PIASxα, PIASxβ, and PIAS4 or PIASγ). The agent may be a small molecule which inhibits a SUMO E3. The agent may be an antibody or an antibody fragment which specifically binds to a SUMO E3. The agent may be an inhibitory nucleic acid as described herein which specifically binds to a nucleic acid encoding a SUMO E3. In certain embodiments, the agent is an inhibitor of PIASγ or PIAS4.

In certain embodiments, the agent is an activator of a sentrin-specific protease (SENP), such as SENP1, SENP2, SENP3, SENP4, SENP5, SENP6, and SENP7.

Also encompassed by the present disclosure is an antibody or a fragment thereof, that specifically binds to a Ras protein, where the antibody or a fragment thereof recognizes an epitope comprising amino acid residue 42 or amino acid residue 104 of the Ras protein. Amino acid residue 42 or amino acid residue 104 of the Ras protein may or may not be sumoylated by a SUMO protein.

The present disclosure provides for an antibody or a fragment thereof, that specifically binds to a Ras protein sumoylated by a SUMO protein. The Ras protein may be sumoylated at amino acid residue 42 or amino acid residue 104.

The present disclosure provides for a method of diagnosing cancer. In certain embodiments, the method comprises the step of contacting a test sample from a subject with the present antibodies, where an increased level of sumoylated Ras protein in the test sample of the subject compared to a control or reference sample indicates that the subject has cancer or is at risk of developing cancer.

The present disclosure provides for a modified Ras protein comprising the amino acid sequence set forth in SEQ ID NO: 2 (or SEQ ID NO: 4, or SEQ ID NO: 6, or SEQ ID NO: 8) comprising a point mutation at amino acid residue 42 or amino acid residue 104 of SEQ ID NO:2 (or SEQ ID NO: 4, or SEQ ID NO: 6, or SEQ ID NO: 8). In certain embodiments, amino acid residue 42 or amino acid residue 104 is substituted with an amino acid other than Lysine. In certain embodiments, amino acid residue 42 and/or amino acid residue 104 are (is) substituted with arginine.

The present disclosure provides for a modified Ras protein comprising the amino acid sequence set forth in SEQ ID NO: 2 (or SEQ ID NO: 4, or SEQ ID NO: 6, or SEQ ID NO: 8) comprising the following mutations: (i) a first point mutation at amino acid residue 42 or amino acid residue 104 of SEQ ID NO:2 (or SEQ ID NO: 4, or SEQ ID NO: 6, or SEQ ID NO: 8) substituted with an amino acid other than Lysine; and (ii) a second point mutation at amino acid residue 12 of SEQ ID NO:2 (or SEQ ID NO: 4, or SEQ ID NO: 6, or SEQ ID NO: 8) substituted with a valine or an amino acid other than glycine, at amino acid residue 13 of SEQ ID NO:2 (or SEQ ID NO: 4, or SEQ ID NO: 6, or SEQ ID NO: 8) substituted with an amino acid other than glycine, at amino acid residue 17 of SEQ ID NO:2 (or SEQ ID NO: 4, or SEQ ID NO: 6, or SEQ ID NO: 8) substituted with asparagine or an amino acid other than serine, at amino acid residue 61 of SEQ ID NO:2 (or SEQ ID NO: 4, or SEQ ID NO: 6, or SEQ ID NO: 8) substituted with an amino acid other than glutamine, and/or at amino acid residue 119 of SEQ ID NO:2 (or SEQ ID NO: 4, or SEQ ID NO: 6, or SEQ ID NO: 8) substituted with asparagine or an amino acid other than aspartic acid. In certain embodiments, amino acid residue 42 or amino acid residue 104 is substituted with an amino acid other than Lysine. In certain embodiments, amino acid residue 42 and/or amino acid residue 104 are (is) substituted with arginine.

In certain embodiments, amino acid residue 42 and/or amino acid residue 104 of the modified Ras protein of the present disclosure is substituted with arginine.

In certain embodiments, the modified Ras protein has a reduced activity compared to the Ras protein without the modification.

The present disclosure provides for a composition comprising the modified Ras protein.

The present disclosure provides for a nucleic acid encoding the present modified Ras protein, a recombinant vector comprising the nucleic acid encoding the present modified Ras protein, and a cell comprising the nucleic acid or the recombinant vector.

The present disclosure provides for an antibody that specifically binds to the modified Ras protein.

The present disclosure provides for a fusion protein comprising a Ras protein having a Flag-tag and a SUMO-3 protein having a human influenza hemagglutinin (HA)-flag.

The present disclosure provides for a cell comprising a vector expressing a Ras protein having a Flag-tag and a SUMO-3 protein having a human influenza hemagglutinin (HA)-flag.

The present disclosure provides for a method for identifying an agent that interferes with sumoylation of a Ras protein by a SUMO protein. In certain embodiments, the method comprises: (i) contacting the Ras protein with a labeled agent in the presence of the SUMO protein; (ii) detecting binding of the labeled agent with the Ras protein or binding of the labeled agent with the SUMO protein; and (iii) isolating the labeled agent that binds to the Ras protein or the SUMO protein.

The present disclosure also provides for a method for increasing sumoylation of a Ras protein by a SUMO protein in a cell comprising introducing an antibody that specifically binds to SENP1 and/or SENP2 in the cell.

In certain embodiments, the Ras protein is HRAS, KRAS or NRAS.

In certain embodiments, the Ras protein is a wild-type Ras protein. In certain embodiments, the Ras protein comprises the amino acid sequence set forth in SEQ ID NO: 2, SEQ ID NO: 4, SEQ ID NO: 6, or SEQ ID NO: 8.

In certain embodiments, the Ras protein is a mutant Ras protein. In certain embodiments, the mutant Ras protein comprises a point mutation. In certain embodiments, the point mutation may be at amino acid residue 12, 13, 17, 35, 59, 60, 61 and/or 119 of the Ras protein. In certain embodiments, the mutant Ras protein is an oncogenic Ras protein. In certain embodiments, the mutant Ras protein is an activated Ras protein which refers to a mutated form of a Ras protein which is capable of inducing cells to lose their normal growth control. In certain embodiments, the Ras protein comprises the amino acid sequence set forth in SEQ ID NO: 2 (or SEQ ID NO: 4, or SEQ ID NO: 6, or SEQ ID NO: 8) comprising the point mutation at amino acid residue 12, 13, 17, 35, 59, 60, 61 and/or 119 of SEQ ID NO:2 (or SEQ ID NO: 4. or SEQ ID NO: 6, or SEQ ID NO: 8).

In certain embodiments, the point mutation of the Ras protein is (i) at amino acid residue 12 which is substituted with valine or an amino acid other than glycine, (ii) at amino acid residue 13 which is substituted with an amino acid other than glycine, (iii) at amino acid residue 17 which is substituted with asparagine or an amino acid other than serine, (iv) at amino acid residue 61 which is substituted with an amino acid other than glutamine, and/or (v) at amino acid residue 119 which is substituted with asparagine or an amino acid other than aspartic acid.

In certain embodiments, amino acid residue 42 or amino acid residue 104 of a Ras protein is substituted with an amino acid other than lysine. In certain embodiments, amino acid residue 42 or amino acid residue 104 of a Ras protein is substituted with arginine.

### 4. BRIEF DESCRIPTION OF THE FIGURES

FIGS. 1A-1F. RAS proteins are post-translationally modified by SUMO3. (FIG. 1A) HEK293 cells were co-transfected with Flag-BRAS and HA-tagged SUMO isoforms as indicated. Equal amounts of protein lysates from various treatments were immunoprecipitated with the anti-Flag antibody. Flag immunoprecipitates, along with lysate inputs, were blotted with either anti-HA antibody or anti-Flag antibody. (FIG. 1B) HEK293 cells were co-transfected with expression constructs of Flag-BRAS, HA-SUMO3, and UBC9 (WT) or UBC9 inactive mutant (mt). Equal amounts of protein lysates from various treatments were immunoprecipitated with the anti-Flag antibody. Flag immunoprecipitates, along with lysate inputs, were blotted with the anti-HA antibody or the anti-Flag antibody. (FIG. 1C) HEK293T cells were co-transfected with constructs expressing Flag-RAS isoforms (HRAS, NRAS, and KRAS4B) and HA-SUMO isoforms (SUMO1, SUMO2, and SUMO3) as indicated. Equal amounts of protein lysates from various treatments were immunoprecipitated with the anti-Flag antibody. Flag immunoprecipitates, along with lysate inputs, were blotted with the anti-Flag antibody or with the anti-HA antibody. (FIG. 1D) HEK293T cell lysates were incubated with bead immobilized with mouse monoclonal anti-RAS IgGs or with control IgGs. Proteins bound to the bead, along the lysate input, were blotted with a rabbit polyclonal anti-RAS antibody or with the ani-SUMO2/3 antibody. Both short (upper panel) and long (lower panel) exposures of RAS blot were shown. Endogenous native RAS and sumoylated RAS are indicated. (FIG. 1E) Pancreatic cell lines including BxPC-3 (wild-type KRAS), MiaPaCa-2 and Panc-1 (KRASV12) were transfected with Flag-KRAS and HA-SUMO3 for 24 h, after which equal amounts of cell lysates were precipitated with the anti-Flag antibody. Flag immunoprecipitates, along with cell lysates, were then blotted with the HA antibody. (FIG. 1F) BxPC-3, MiaPaCa-2 and Panc-1 cells were lysed and equal amounts of cell lysates were incubated with bead immobilized with mouse monoclonal anti-RAS IgGs or with control IgGs. Proteins bound to the bead, along the lysate inputs, were blotted with a rabbit polyclonal anti-KRAS4B antibody. Both endogenous native KRAS and sumoylated KRAS are indicated.

FIGS. 2A-2E. Lysine 42 is a primary site for sumoylation. (FIG. 2A) MiaPaCa-2 cells were transfected with Flag-KRAS and HA-SUMO3 expression constructs for 24 h and then treated with or without 2-D08 for 18 h. Cells were then lysed and equal amounts of the lysates were immunoprecipitated with the anti-Flag antibody. Flag precipitates, along with lysate inputs, were blotted with antibodies to HA and Flag, respectively. (FIG. 2B) HEK293T cells were co-transfected with plasmid constructs expressing Flag-KRAS, HA-SUMO3, and SENP isoforms as indicated. Equal amounts of protein lysates were immunoprecipitated with the anti-Flag antibody. Flag immunoprecipitates were immunoblotted with antibodies to Flag or HA. Protein lysates of various treatments were also blotted with antibodies to HA, Flag, β-actin, SENP 1, SENP2, or SENP6. (FIG. 2C) Alignment of RAS isoform amino acid sequences with a predicted sumoylation site (K42). The residue for optimal sumoylation was predicted with SUMOplot (http://www.abgent.com/sumoplot). (FIG. 2D) HEK293T cells were co-transfected with constructs expressing Flag-tagged HRAS (WT) or HRASK42R, HA-UBC9, HA-SUMO3. Equal amounts of protein lysates from various treatments were immunoprecipitated with the anti-Flag antibody. Flag immunoprecipitates, along with lysate inputs, were blotted with the anti-Flag antibody or with the anti-HA antibody. (FIG. 2E) HEK293T cells were co-transfected with plasmid constructs expressing Flag-HRAS or various mutants as indicated (HRAS42R, HRAS104R, or HRAS42R/104R), and HA-SUMO3 for 24 h. Equal amounts of protein lysates from various transfection were immunoprecipitated with the anti-Flag antibody. Flag immunoprecipitates, along with lysate inputs, were blotted with the anti-Flag antibody or with the anti-HA antibody.

FIGS. 3A-3E. PIASγ is SUMO E3 ligase for RAS proteins. (FIG. 3A) HEK293T cells were co-transfected with plasmid constructs expressing Flag-tagged proteins of the PIAS family, Flag-KRAS and/or HA-SUMO3. Equal amounts of protein lysates from various treatments were immunoprecipitated with the anti-Flag antibody. Flag immunoprecipitates, along with the lysate inputs, were immunoblotted with the anti-Flag or the anti-HA antibody. (FIG. 3B) HEK293T cells were co-transfected with individual Flag-tagged PIAS expression constructs and GFP-HRAS expression construct. Equal amounts of protein lysates were immunoprecipitated with the anti-Flag antibody. Flag immunoprecipitates, along with lysate inputs, were immunoblotted with an anti-GFP antibody or anti-Flag antibody. (FIG. 3C) HEK293T cells were co-transfected with plasmid constructs expressing Flag-PIASγ (or vector) and UBC9. Equal amounts of protein lysates were immunoprecipitated with the anti-Flag antibody. Flag immunoprecipitates, along with lysate inputs, were blotted with antibodies to Flag and RAS. Endogenous RAS and IgGs (light and heavy chains) are indicated. (FIG. 3D) HEK293T cells were transfected with plasmid constructs expressing HA-SUMO3, Flag-KRAS, PIASγ, and/or UBC9 for 24 h after which cells were collected and lysed. Equal amounts of cell lysates were immunoprecipitated with the Flag antibody. Immunoprecipitates, along with lysate inputs, were blotted with antibodies to HA, Flag, PIASg, and/or UBC9. (FIG. 3E) HEK293T cells were co-transfected with plasmid constructs expressing Flag-HRAS mutants (N17 or V12) and HA-SUMO3 [or HA-ubiquitin (Ub)] for 24 h after which cells were collected for lysate preparation. Equal amounts of cell lysates from various transfection were immunoprecipitated with the Flag antibody. Flag immunoprecipitates, along with cell lysate inputs, were blotted with antibodies to Flag and HA.

FIGS. 4A-4F. Sumoylation is involved in RAS activation. (FIG. 4A) HEK293T cells were co-transfected with HA-SUMO3 construct and Flag-tagged constructs in fasting and growing cells. (FIG. 4B) HEK293T cells were co-transfected with HA-SUMO3 construct and Flag-tagged constructs expressing WT KRAS, KRAS42R, KRASV12, or KRASV12/42R for 24 h, after which cells were collected for lysate preparation. Equal amounts of lysates were then precipitated with the Flag antibody. Flag immunoprecipitates, along with lysate inputs, were blotted with the antibodies to HA and Flag. (FIG. 4C) HEK293T cells were co-transfected plasmid constructs expressing Flag-KRAS (either WT or V12) and HA-SUMO3 for 24 h after which cells were collected and lysed. Equal amounts of cell lysates were precipitated with the anti-Flag antibody. Flag immunoprecipitates were blotted with the anti-Flag or anti-HA antibody. Lysates were also blotted with antibodies to Flag, phospho-ERK (p-ERK), and b-actin. (FIG. 4D HEK293T cells were co-transfected plasmid constructs expressing Flag-KRASV12 (or KRASV12K42R) and HA-SUMO3 for 24 h, after which cell lysates were immunoprecipitated with the anti-Flag antibody. Flag immunoprecipitates were blotted with the anti-Flag antibody or the anti-HA antibody. Lysates were also blotted with antibodies to p-ERK, total ERK, Flag, and α-tubulin. (FIG. 4E) Tet293/KRASV12 or Tet293/KRASV12/42R cells were cultured in the presence or absence of Dox. At various times of culture, cells were collected and lysed. Equal amounts of cell lysates were blotted with antibodies to Flag (KRAS), pan-RAS, phospho-cRaf338 (p-cRaf388), phospho-MEK (p-MEK), pan-MEK, p-ERK, pan-ERK, phospho-AKT (p-AKT), pan-AKT, and b-actin. (FIG. 4F HEK293T cells were co-transfected with plasmid constructs expressing Flag-KRASV12 or Flag-KRASV12/42R and GFP-Rafl-RBD or GFP-Ral.GDS-RBD for 24 h. The vector plasmid was also used as control. After transfection, cells were collected and lysed. Equal amounts of lysates were immunoprecipitated with the Flag antibody. Immunoprecipitates, along with lysate inputs, were blotted with antibodies to GFP and Flag, respectively.

FIGS. 5A-5I. Sumoylation is essential for KRAS oncogenic activities in vitro. (FIG. 5A) NIH3T3 cells transfected with Flag-KRASV12, Flag-KRASV12/42R expression plasmid, or vector alone for 24 h were subjected to conventional wound-healing assays. Representative images of cells at 0 and 18 h post scratching were shown. (FIG. 5B) Percent of wound closure as described in A was quantified. Data were summarized from three independent experiments. Ve stands for vector-transfected control. (FIG. 5C) Equal amounts of lysates from cells transfected with various expression plasmids as described in B were blotted with antibodies to Flag and b-actin, respectively. (FIG. 5D) MCF cells were transfected with a plasmid construct expressing Flag-KRASV12 or Flag-KRASV12/42R for 24 h. Empty plasmid vector was also used for transfection as control. Images at 18 h post scratching for various transfections were taken. Representative images were shown. (FIG. 5E) MCF7 cells were transfected with a plasmid construct expressing KRASV12 or KRASV12/42R for 24 h. Empty plasmid vector was also used for transfection as control. Equal amounts of cell lysates were blotted for pcRaf388, p-MEK, MEK, p-ERK, ERK, Snail, claudin-1, Flag, and b-actin. (FIG. 5F) Tet293 cells stably transfected with KRAS V12 or KRASV12/42R expression plasmid were cultured in the presence of Dox for 24 h after which treated cells, along with parental cells, were used for transwell migration assays. Representative images were shown. (FIG. 5G) Cell invasiveness as described in F was quantified. Data were summarized from 3 independent experiments. Ve stands for vector-transfected control. (FIG. 5H) NIH3T3 cells were transfected with a plasmid construct expressing KRASV12 or KRASV12/42R for 24 h, after which transfected cells, along with cells transfected with vector alone, were subjected to transwell migration assays. Representative images were shown. (FIG. 5I) Tet293 cells stably transfected with a plasmid construct expressing KRASV12 or KRASV12/42R were cultured in the presence of Dox for various times as indicated, after which cells were collected and lysed. Equal amounts of cell lysates were blotted with antibodies to Snail and b-actin, respectively.

FIGS. 6A-6C. Sumoylation is important for KRAS oncogenic activities in vivo. (FIG. 6A) Athymic nude mice were inoculated subcutaneously with Tet293 cells stably expressing KRASV12 or KRASV12/42R or inoculated with parental HEK293T cells as control. Doxocycline was injected peritoneally after inoculation and subsequently administrated into mice every 3 days. The size (diameter) of tumors were measured on weekly schedules. Representative mice with tumors and tumor samples from each group are shown. (FIG. 6B) Data were summarized from the mouse xenograft experiments. The error bars represent SD. (FIG. 6C) Sections of xenograft tumor samples were stained with antibodies to phospho-ERK, phospho-AKT, Ki-67, and PCNA, respectively. Representative images are shown.

FIGS. 7A-7E. SUMO inhibitor blocks migration of pancreatic cancer cells with KRASV12 mutation. (FIG. 7A) MiaPaCa-2 cells transfected with various KRAS expression constructs or the vector as indicated for 24 h. A GFP-expressing plasmid was used for co-transfection. Transfected cells were then used for wound-healing assays as described in Methods. Representative images of the assay and GFP expression are shown. (FIG. 7B) Both BxPC-3 and MiaPaCa-2 cells were employed for wound-healing assays. Cells with open scratches were treated with various concentrations of 2-D08 for 18 h. Representative images of cells before and after recovery in the presence of 2-D08 for 18 h are shown. (FIG. 7C & FIG. 7D) Data were summarized from experiments as shown in A and B, respectively. (FIG. 7E) MiaPaCa-2 cells treated with or without 2-D08 were lysed and equal amounts of cell lysates were blotted with antibodies to N-Cadherin, ZEB1/TCF8, ZO-1, PARP-1 and B-actin, respectively.

FIG. 8. Nucleic acid sequences of H-RAS (SEQ ID NO:1); N-RAS (SEQ ID NO:3); K-RAS 4A (SEQ ID NO:5); KRAS 4B (SEQ ID NO:7).

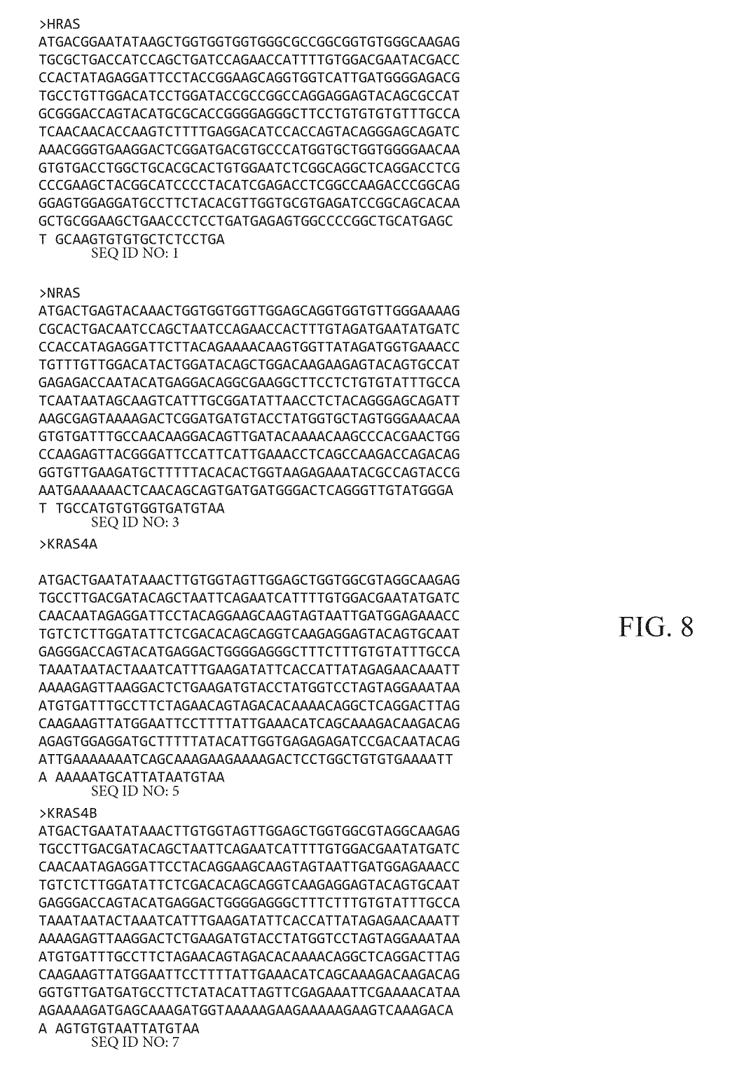

FIG. 9. Amino acid sequences of H-RAS (SEQ ID NO:2); N-RAS (SEQ ID NO:4); K-RAS 4A (SEQ ID NO:6); KRAS 4B (SEQ ID NO:8).

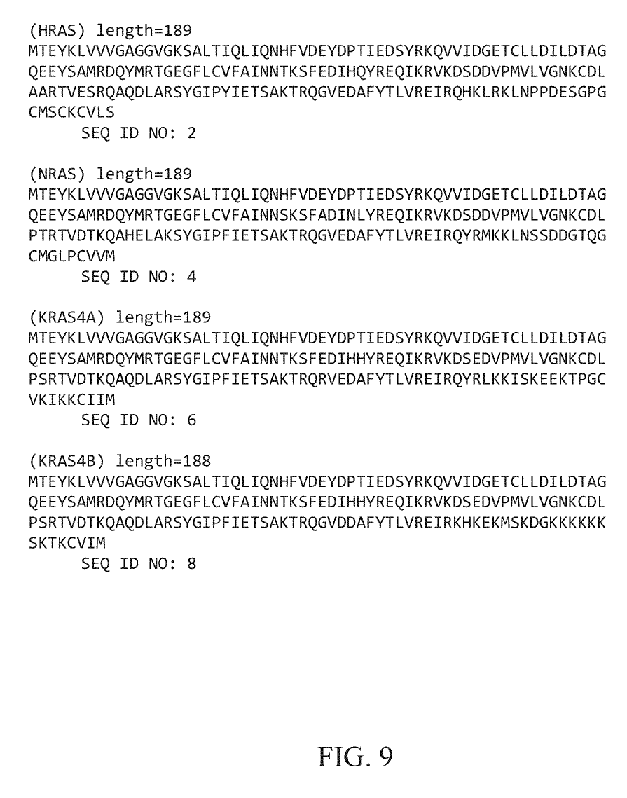

FIGS. 10A-10B. Sumoylation-resistant RAS mutant fails to concentrate at the plasma membrane. (FIG. 10A) HeLa cells were co-transfected with CFP-H-RAS and GFP-Rafl RBD (RAS Binding Domain) for 24 h, after which cells were serum-starved for 24 h followed with the addition of 20% FBS for 10 min. Cells were then fixed and examined by fluorescence microscopy. (FIG. 10B) HeLa cells were co-transfected with plasmid constructs expressing CFP-RAS (or its mutant K42R) and GFP-Rafl RBD for 48 h after which cells were fixed and examined by fluorescence microscopy.

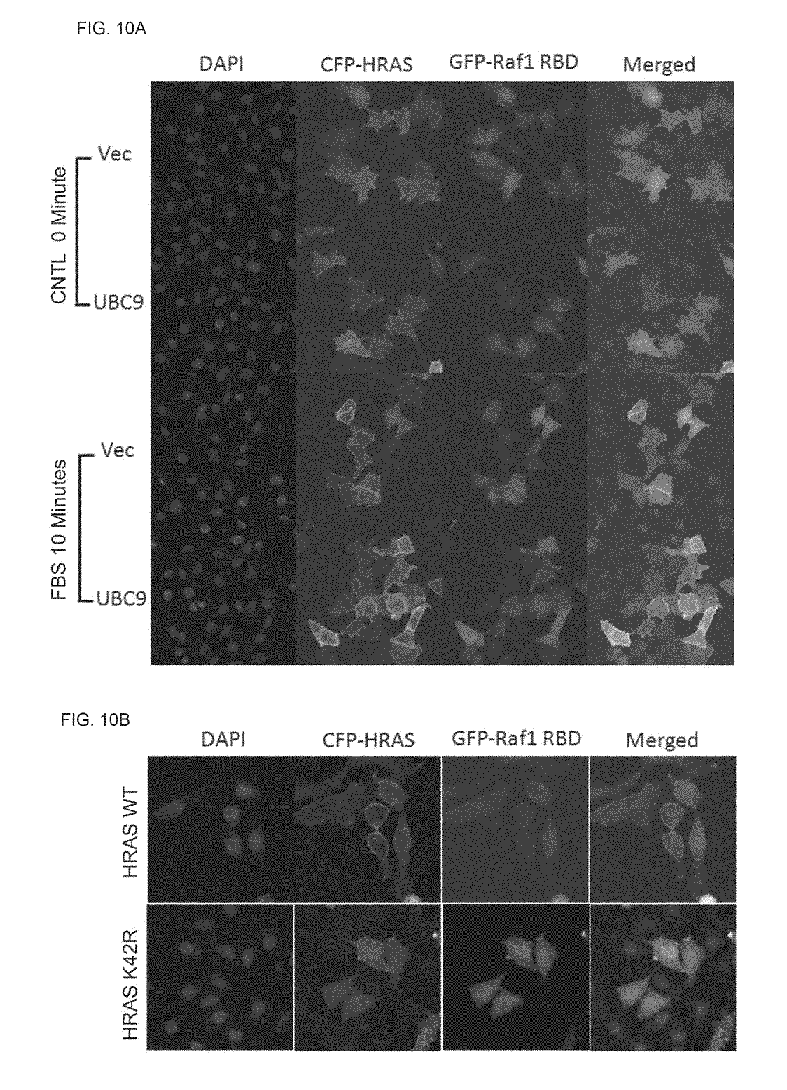

FIG. 11. HCT116 Cells were transfected with a plasmid construct expressing KRASV12 or KRASV12K42R, or with vector control, for various times. Cell numbers were determined and summarized.

FIG. 12. HEK293 cells were transfected with Flag-tagged plasmid constructs expressing individual SUMO E3 ligases of the PIAS family (PIAS1, PIAS3, PIASxa, PIASxb, and PIASγ) for 24 h after which cells were collected and lysed. Equal amounts of lysates were immunoprecipitated with the anti-Flag antibody. Flag immunoprecipitates, along with lysate inputs, were blotted with the anti-RAS antibody.

FIG. 13. Wild-Type Human SUMO amino acid sequences: SUMO-1 (SEQ ID NO: 9); SUMO-2 (SEQ ID NO: 10); SUMO-3 (SEQ ID NO:11); and SUMO-4 (SEQ ID NO:12).

### 4.1 Definitions

As used herein, the terms “RAS” and “RAS protein” refer to any protein within the RAS family, including, but not limited to, any wild-type or mutant forms, whether naturally occurring or engineered, of the RAS protein. Ras family members include, but are not limited to, HRAS; KRAS; NRAS; KRAS 4A; KRAS 4B; DIRAS1; DIRAS2; DIRAS3; ERAS; GEM; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; and RRAS2. Wennerberg et al., (2005) The Ras superfamily at a glance. J. Cell. Sci. 118 (5): 843-6.

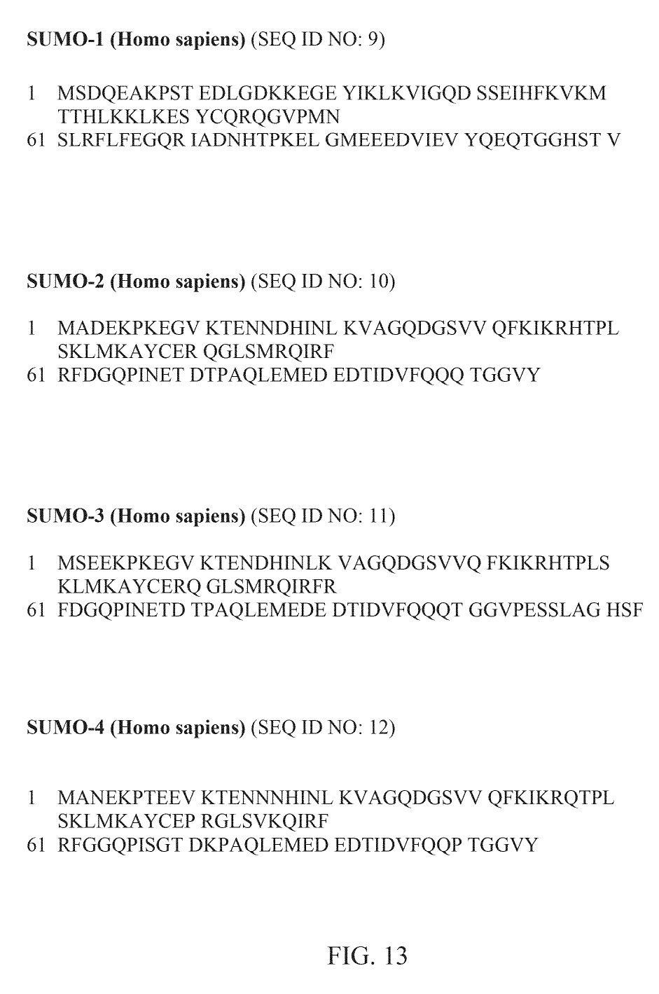

As used herein, the term “SUMO” is intended to refer to a small ubiquitin-like modified protein, a polypeptide or fragment thereof, encoded by a SUMO gene. Examples of Wild-type (WT) human SUMO proteins include the SUMO protein isoforms known as SUMO-1, SUMO-2, SUMO-3 and SUMO-4, as illustrated in FIG. 13 (SEQ ID NO: 9; SEQ ID NO: 10; SEQ ID NO: 11; and SEQ ID NO: 12). Human SUMO-1 and SUMO-2 are identical to those of murine SUMO-1 and SUMO-2. SUMO2 and 3 share 96% sequence homology whereas 52% is obtained between SUMO1 and SUMO3. The polypeptide sequence of murine SUMO-3 differs from human SUMO-3 at position 93-103, although residues 1-92 are identical.

As used herein, the term “sumoylation” refers to the binding of SUMO protein to a target protein, e.g., a Ras protein, which may change the activities of the target protein.

As used herein, the term “SUMOylation site” is intended to mean a site in the target protein that reacts with a SUMO protein. In certain embodiments, the SUMOylation site is a Type I (consensus) site and Type II (non-consensus) sites. Type I sites followed the ψKXE (ψ is A, I, L, M, P, F, or V and X is any amino acid residue) motif [Geiss-Friedlander, R., Melchior, F., Concepts in sumoylation: a decade on. Nat. Rev. Mol. Cell Biol. 2007, 8, 947-956; Rodriguez, M. S., Dargemont, C., Hay, R. T., SUMO-1 conjugation in vivo requires both a consensus modification motif and nuclear targeting. J. Biol. Chem. 2001, 276, 12654-126591 In certain embodiments, the SUMOylation site is a Type II site containing other non-canonical sites.

As used herein, the term “protein” includes polypeptides, peptides, fragments of polypeptides, and fusion polypeptides.

As used herein, the term “modulating” and “modulation” refer to alteration of the activity of a protein as detected by methods known in the art.

As used herein, the term “modulator” refers to any substance (e.g., small molecules, compounds, peptide, protein, etc.) which alters the activity of a protein as detected by methods known in the art.

As used herein, the terms “treatment” and “treating” refer to administering a composition of the invention to effect an alteration or improvement of the disease or condition. Prevention, amelioration, and/or treatment can require administration of multiple doses at regular intervals, or prior to onset of the disease or condition to alter the course of the disease or condition. Moreover, a single agent can be used in a single individual for each prevention, amelioration, and treatment of a condition or disease sequentially, or concurrently.

“Therapeutically effective amount” means an amount of a pharmaceutical agent that provides a therapeutic benefit to a subject. It also refers to an amount of a compound that produces a desired effect. For example, a population of cells may be contacted with an effective amount of a compound to study its effect in vitro (e.g., cell culture) or to produce a desired therapeutic effect ex vivo or in vitro. An effective amount of a compound may be used to produce a therapeutic effect in a subject, such as preventing or treating a target condition, alleviating symptoms associated with the condition, or producing a desired physiological effect. In such a case, the effective amount of a compound is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to, the characteristics of the compound (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the compound is administered alone or in combination with another compound, drug, therapy or other therapeutic method or modality. One skilled in the clinical and pharmacological arts will be able to determine an effective amount or therapeutically effective amount through routine experimentation, namely by monitoring a cell's or subject's response to administration of a compound and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein.

The terms “antagonist” and “inhibitor” are used interchangeably, and they refer to a compound having the ability to inhibit a biological function of a target protein, whether by inhibiting the activity or expression of the protein, such as K-Ras, H-Ras or N-Ras. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

The term “selective inhibition” or “selectively inhibit” refers to a biologically active agent refers to the agent's ability to preferentially reduce the target signaling activity as compared to off-target signaling activity, via direct or indirect interaction with the target.

A “pharmaceutical compositions” mean a mixture of substances suitable for administering to a subject. Pharmaceutical compositions can comprise, for example, a combination of pharmaceutical agents as well as the presence of a sterile aqueous solution or other standard pharmaceutical additive known in the art.

“Administering” means providing a pharmaceutical agent or composition to a subject, and includes, but is not limited to administering by a medical professional and self-administering. Co-administration is the administration of two or more pharmaceutical agents to an animal. The two or more pharmaceutical aunts can be in a single pharmaceutical composition, or can be in separate pharmaceutical compositions. Each of the two or more pharmaceutical agents can be administered through the same or different routes of administration. Co-administration encompasses administration in parallel, concomitant or sequentially.

As used herein, the term “subject” refers to an animal, including, but not limited to a human, to whom a pharmaceutical composition is administered. Animals include humans or non-human animal, including, but not limited to, mice, rats, rabbits, dogs, cats, pigs, and non-human primates, including, but not limited to, monkeys and chimpanzees.

As used herein, the term “cell proliferation” refers to a phenomenon by which the cell number has changed as a result of division. This term also encompasses cell growth by which the cell morphology has changed (e.g., increased in size) consistent with a proliferative signal.

### 5. DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, methods, apparatuses, or systems that would be known by one of ordinary skill in the art have not been described in detail so as not to obscure claimed subject matter. It is to be understood that particular features, structures, or characteristics described may be combined in various ways in one or more implementations.

The present disclosure provides a method for modifying (e.g., reducing or enhancing) the activity of a Ras protein in a cell. The method may contain the step of contacting the cell with an agent that interferes with sumoylation of the Ras protein at amino acid residue 42 and/or amino acid residue 104 by a small ubiquitin-like modifier (SUMO) protein.

The present disclosure also provides a method for decreasing proliferation and/or migration of a cancer cell. The method may contain the step of contacting the cell with an agent that interferes with sumoylation of the Ras protein at amino acid residue 42 and/or amino acid residue 104 by a small ubiquitin-like modifier (SUMO) protein.

The present disclosure provides a method of treating cancer in a subject. The method may contain the step of administering to the subject an agent that interferes with sumoylation of the Ras protein at amino acid residue 42 and/or amino acid residue 104 by a small ubiquitin-like modifier (SUMO) protein.

Also encompassed by the present disclosure is a method for decreasing sumoylation of a Ras protein by a small ubiquitin-like modifier (SUMO) protein. The method may contain the step of introducing a first point mutation at amino acid residue 42 and/or amino acid residue 104 of the Ras protein to form a modified Ras protein.

The present method may modify (e.g., reduce or enhance) the activity of at least one downstream Ras effector as described herein.

The present method may modify (e.g., reduce or enhance) cell proliferation and/or migration.

The SUMO protein may be SUMO1, SUMO2, SUMO3 or SUMO4. In certain embodiments, the SUMO protein is SUMO3.

The agents for use in the present methods and compositions can be any of a small molecule, an antibody, an antibody fragment, a polypeptide, a carbohydrate, an inhibitory nucleic acid, or any combination thereof.

In certain embodiments, the agent is an antibody or a fragment thereof.

In certain embodiments, the antibody or a fragment thereof specifically binds to an epitope comprising amino acid residue 42 or amino acid residue 104 of the Ras protein. In certain embodiments, the antibody or a fragment thereof specifically binds to an epitope comprising amino acid residue 42 or amino acid residue 104 of the Ras protein, where the Ras protein is sumoylated at amino acid residue 42 or amino acid residue 104.

In certain embodiments, the agent is a SUMO E 1 inhibitor, such as an inhibitor of SAE (e.g., SAE1 or SAE2). The agent may be a small molecule which inhibits a SUMO E1. The agent may be an antibody or an antibody fragment which specifically binds to a SUMO E1. The agent may be an inhibitory nucleic acid as described herein which specifically binds to a nucleic acid encoding a SUMO E1. Non-limiting examples of SUMO E1 inhibitors include the compounds disclosed in U.S. Patent Publication No. 2013/0245032, Kumar et al., Identification of sumoylation activating enzyme 1 inhibitors by structure-based virtual screening, J. Chem. Inf. Model, 2013, 53(4):809-820, Kumar et al., Identification of new SUMO activating enzyme 1 inhibitors using virtual screening and scaffold hopping, J. Chem. Inf. Model, 2013, 53(4):809-820, the contents of each of which are incorporated herein by reference.

In certain embodiments, the agent is a SUMO E2 inhibitor, such as an inhibitor of UBC9. The agent may be a small molecule which inhibits a SUMO E2. The agent may be an antibody or an antibody fragment which specifically binds to a SUMO E2. The agent may be an inhibitory nucleic acid as described herein which specifically binds to a nucleic acid encoding a SUMO E2. Non-limiting examples of SUMO E2 inhibitors include 2-(2,3,4-Trihydroxyphenyl)-4H-chromen-4-one (2-D08, also named 2-(2,3,4-Trihydroxyphenyl)-4H-1-Benzopyran-4-one or 2′,3′,4′-trihydroxy-flavone), and spectomycin B1. Non-limiting examples of SUMO E2 inhibitors also include the compounds disclosed in Hirohama et al., Spectomycin B1 as a Novel SUMOylation Inhibitor That Directly Binds to SUMO E2, ACS Chem. Biol., 2013, 8(12):2635-2642; and Hewitt et al., Insights Into the Allosteric Inhibition of the SUMO E2 Enzyme Ubc9, Angewandte Chemie Int. Ed. Engl., 2016, 55(19), 5703-5707, the contents of each of which are incorporated herein by reference.

In some embodiments, the SUMO E2 inhibitor is 2-(2,3,4-Trihydroxyphenyl)-4H-chromen-4-one (2-D08) including derivatives, analogs, chemical analogs, variants, salts, hydrates, solvates, complexes, and modifications thereof.

The term “derivative” may refer to any derivative of the present agent. In certain embodiments, a derivative of a compound is a chemical substance related structurally to the compound and theoretically derivable from it. In certain embodiments, a derivative of a compound is a substance that can be made from the compound. In certain embodiments, a derivative of a compound is a variant of the compound. In certain embodiments, a derivative of a compound is an analog of the compound. In certain embodiments, a derivative of a compound is a chemical analog of the compound. In certain embodiments, an analog of a compound is a substance that is structurally similar to the compound but differs slightly in composition (e.g., as in the replacement of one atom by an atom of a different element or in the presence of a particular functional group). In certain embodiments, the derivative is a compound (e.g., a drug precursor or a prodrug) that is transformed in vivo to yield the present agent or a pharmaceutically acceptable salt, hydrate or solvate of the compound. The transformation may occur by various mechanisms (e.g., by metabolic or chemical processes), such as, for example, through hydrolysis in blood. Bioreversible Carriers in Drug Design, ed. Edward B. Roche, American Pharmaceutical Association and Pergamon Press, 1987.

The present agent may be in a salt form. As used herein, a “salt” is salt of the instant compounds which has been modified by making acid or base, salts of the compounds. The salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately treating a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, the term “solvate” refers to a complex of variable stoichiometry formed by a solute (e.g., the present agent) and a solvent. Such solvents may not interfere with the biological activity of the solute. Examples of suitable solvents include, but are not limited to, water, acetone, methanol, ethanol and acetic acid. Preferably the solvent is a pharmaceutically acceptable solvent. Non-limiting examples of suitable pharmaceutically acceptable solvents include water, ethanol and acetic acid.

If a chiral center or another form of an isomeric center is present in a compound of the present disclosure, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The present compounds may be in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form. When the structure of the compounds used in this invention includes an asymmetric carbon atom such compound can occur as racemates, racemic mixtures, and isolated single enantiomers. Each stereogenic carbon may be of the R or S configuration. It is to be understood accordingly that the isomers arising from such asymmetry (e.g., all enantiomers and diastereomers) are included within the scope of this invention, unless indicated otherwise.

The present disclosure is also intended to include use of all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include carbon-13 and carbon-14. It will be noted that any notation of a carbon in structures throughout this application, when used without further notation, are intended to represent all isotopes of carbon, such as 12C, 13C, or 14C. Furthermore, any compounds containing 13C or 14C may specifically have the structure of any of the compounds disclosed herein. It will also be noted that any notation of a hydrogen in structures throughout this application, when used without further notation, are intended to represent all isotopes of hydrogen, such as 1H, 2H, or 3H. Furthermore, any compounds containing 2H or 3H may specifically have the structure of any of the compounds disclosed herein.

In certain embodiments, the agent is a SUMO E3 inhibitor, such as an inhibitor of a PIAS protein (e.g., PIAS1, PIAS2, PIASxα, PIASxβ, PIAS3, and PIAS4 (PIASγ)), human polycomb 2 homolog (PC2), histone deacetylase 4 (HDAC4), topoisomerase I-binding RING finger protein (TOPORS) and Ras homolog enriched in striatum (RHES), Cst9, Mms21, Siz1, Siz2, RanBP2, Xip3. Gareau et al, The SUMO pathway: emerging mechanisms that shape specificity, conjugation and recognition, Nat. Rev. Mol. Cell Biol. 2010, 11(12):861-871. The agent may be a small molecule which inhibits a SUMO E3. The agent may be an antibody or an antibody fragment which specifically binds to a SUMO E3. The agent may be an inhibitory nucleic acid as described herein which specifically binds to a nucleic acid encoding a SUMO E3. In certain embodiments, the agent is an inhibitor of PIASγ.

In certain embodiments, the agent is an activator of a sentrin-specific protease (SENP), such as SENP1 and SENP2.

Also encompassed by the present disclosure is an antibody or a fragment thereof, that specifically binds to a Ras protein, where the antibody or a fragment thereof recognizes an epitope comprising amino acid residue 42 or amino acid residue 104 of the Ras protein. Amino acid residue 42 or amino acid residue 104 of the Ras protein may or may not be sumoylated by a SUMO protein.

The present disclosure provides for an antibody or a fragment thereof, that specifically binds to a Ras protein sumoylated by a SUMO protein. The Ras protein may be sumoylated at amino acid residue 42 or amino acid residue 104.

The present disclosure provides for a method of diagnosing cancer by contacting a test sample from a subject with the present antibodies. In certain embodiments, the method comprises the step of diagnosing cancer when detecting an increased level of sumoylated Ras protein in the test sample of the subject compared to a control or reference sample.

The present disclosure provides for a modified Ras protein comprising the amino acid sequence set forth in SEQ ID NO: 2 (or SEQ ID NO: 4, or SEQ ID NO: 6, or SEQ ID NO: 8) comprising a point mutation at amino acid residue 42 or amino acid residue 104 of SEQ ID NO:2 (or SEQ ID NO: 4, or SEQ ID NO: 6, or SEQ ID NO: 8). In certain embodiments, amino acid residue 42 or amino acid residue 104 is substituted with an amino acid other than Lysine. In certain embodiments, amino acid residue 42 is substituted with arginine.

The present disclosure provides for a modified Ras protein comprising the amino acid sequence set forth in SEQ ID NO: 2 (or SEQ ID NO: 4, or SEQ ID NO: 6, or SEQ ID NO: 8) comprising the following mutations: (i) a first point mutation at amino acid residue 42 or amino acid residue 104 of SEQ ID NO:2 (or SEQ ID NO: 4, or SEQ ID NO: 6, or SEQ ID NO: 8) substituted with an amino acid other than Lysine; and (ii) a second point mutation at amino acid residue 12 of SEQ ID NO:2 (or SEQ ID NO: 4, or SEQ ID NO: 6, or SEQ ID NO: 8) substituted with a valine or an amino acid other than glycine, at amino acid residue 13 of SEQ ID NO:2 (or SEQ ID NO: 4, or SEQ ID NO: 6, or SEQ ID NO: 8) substituted with an amino acid other than glycine, at amino acid residue 17 of SEQ ID NO:2 (or SEQ ID NO: 4, or SEQ ID NO: 6, or SEQ ID NO: 8) substituted with asparagine or an amino acid other than serine, at amino acid residue 61 of SEQ ID NO:2 (or SEQ ID NO: 4, or SEQ ID NO: 6, or SEQ ID NO: 8) substituted with an amino acid other than glutamine, and/or at amino acid residue 119 of SEQ ID NO:2 (or SEQ ID NO: 4, or SEQ ID NO: 6, or SEQ ID NO: 8) substituted with asparagine or an amino acid other than aspartic acid. In certain embodiments, amino acid residue 42 or amino acid residue 104 is substituted with an amino acid other than Lysine. In certain embodiments, amino acid residue 42 is substituted with arginine.

The present disclosure provides for a nucleic acid encoding the present modified Ras protein, a recombinant vector comprising the nucleic acid encoding the present modified Ras protein, and a cell comprising the nucleic acid or the recombinant vector.

In certain embodiments, the Ras protein is a wild-type Ras protein. In certain embodiments, the Ras protein comprises the amino acid sequence set forth in SEQ ID NO: 2, SEQ ID NO: 4, SEQ ID NO: 6, or SEQ ID NO: 8.

In certain embodiments, the Ras protein is a mutant Ras protein. In certain embodiments, the mutant Ras protein comprises a point mutation. In certain embodiments, the point mutation may be at amino acid residue 12, 13, 17, 35, 59, 60, 61 and/or 119 of the Ras protein. In certain embodiments, the mutant Ras protein is an oncogenic Ras protein. In certain embodiments, the mutant Ras protein is an activated Ras protein which refers to a mutated form of a Ras protein which is capable of inducing cells to lose their normal growth control. In certain embodiments, the Ras protein comprises the amino acid sequence set forth in SEQ ID NO: 2 (or SEQ ID NO: 4, or SEQ ID NO: 6, or SEQ ID NO: 8) comprising the point mutation at amino acid residue 12, 13, 17, 35, 59, 60, 61 and/or 119 of SEQ ID NO:2 (or SEQ ID NO: 4, or SEQ ID NO: 6, or SEQ ID NO: 8).

In certain embodiments, the point mutation of the Ras protein is (i) at amino acid residue 12 which is substituted with valine or an amino acid other than glycine, (ii) at amino acid residue 13 which is substituted with an amino acid other than glycine, (iii) at amino acid residue 17 which is substituted with asparagine or an amino acid other than serine, (iv) at amino acid residue 61 which is substituted with an amino acid other than glutamine, and/or (v) at amino acid residue 119 which is substituted with asparagine or an amino acid other than aspartic acid.

In certain embodiments, amino acid residue 42 or amino acid residue 104 is substituted with an amino acid other than lysine. In certain embodiments, amino acid residue 42 or amino acid residue 104 is substituted with arginine.

RAS proteins are GTPases that participate in multiple signal cascades, regulating crucial cellular processes including cell survival, proliferation, differentiation, and autophagy. The Ras family of small GTPases is composed of intracellular signaling molecules which are regulated by the nucleotide guanosine-5′-triphosphate (GTP). These proteins function as binary signaling switches with both “on” and “off” states. In the “off” state, Ras is bound to the nucleotide guanosine diphosphate (GDP), while in the “on” state, Ras is bound to GTP. Hence, activation and deactivation of Ras are controlled by cycling between the active GTP-bound and inactive GDP-bound forms. In the GTP-bound conformation, Ras has high affinity for numerous effectors which allow it to carry out its intracellular functions. Ras-regulated signaling pathways control processes as diverse as actin cytoskeletal integrity, proliferation, differentiation, cell adhesion, apoptosis, and cell migration. Mutations or deregulated activities of RAS are frequently the driving force for oncogenic transformation and tumorigenesis. Described herein is that crucial roles of sumoylation in controlling stability, activity, and/or subcellular localization of many important proteins, key cellular regulators that involved in proliferation, DNA damage responses, and chromatin remodeling, it is investigated whether RAS proteins were post-translationally modified by SUMO.

Ras proteins consist of three contiguous regions. The first region encompasses the N-terminal 86 amino acids, which are 100% identical among the different Ras proteins. The next 80 amino acids define a second region where mammalian Ras proteins diverge only slightly from each other, exhibiting an 85% homology between any protein pair. The remaining C-terminal sequence, known as the hypervariable region, starts at amino acid 165 and shows no sequence similarity among Ras proteins except for a conserved CAAX motif (C, cysteine; A, aliphatic amino acid; X, methionine or serine) at the very C-terminal end, which is present in all Ras proteins and directs posttranslational processing (Bar-Sagi, 2001, Mol. Cell Biol., 21(5): 1441-1443). References to particular amino acid residue numbers in the Ras protein primary structure refer to the residue number corresponding to the aligned amino acid sequences of several Ras proteins. Alignment of Ras proteins for purposes of determining amino acid sequence residue number can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In certain embodiments, a mutant Ras protein is any of the mutant Ras proteins. In certain embodiments, a mutant Ras protein is a constitutively active mutant Ras protein. In one embodiment, a mutant Ras protein is a constitutively active mutant Ras protein with the inability to adopt its “off” state, irrespective of whether it is bound to GDP. Consequently, aberrant signaling through the Ras pathway can play an important role in uncontrolled cell proliferation and tumorigenesis. In certain embodiments, a mutant Ras protein is unable to hydrolyze GTP. In certain embodiments, the mutant Ras protein is unable to bind GDP. In certain embodiments, a mutant Ras protein has one or more mutations at amino acid residue(s) known to contact one or more downstream Ras effector proteins. In certain embodiments, a mutant Ras protein has one or more mutations at amino acid residue(s) 12, 13 17, 35, 59, 60, 61, 119, or a combination thereof. In certain embodiments, a mutant Ras protein has one or more mutations at amino acid residue(s) G12, G13, S17, T35, G60, Q61, D119, or a combination thereof. The substitution mutations include, but are not limited to, G12V, G12D, G12C, G13D, S17N, Q61H, D119N, and combinations thereof of a Ras protein. Lu et al., Cancer Res. 2004, 64(15):5084-8. Abrams et al, Semin. Oncol, 23(1):118-134. U.S. Patent Publication No. 20150051110. In certain embodiments, the mutant Ras protein is an oncogenic Ras protein. In certain embodiments, the mutant Ras protein is an activated Ras protein which refers to a mutated form of a Ras protein which is capable of inducing cells to lose their normal growth control.

As used herein, a “mutation” includes an amino acid residue deletion, an amino acid residue insertion, and/or an amino acid residue substitution of at least one amino acid residue in a defined primary amino acid sequence, such as a primary amino acid sequence of a Ras protein. An amino acid “substitution” means that at least one amino acid residue of a defined primary amino acid sequence (such as a primary amino acid sequence of a Ras protein) is replaced with another amino acid.

In certain embodiments, the mutations in the Ras protein amino acid sequence prevent the protein from adopting an active conformation (i.e., the mutations result in a constitutively inactive Ras protein). In yet other aspects, the mutations in the Ras protein amino acid sequence do not completely abolish the ability of the protein to switch from one conformational state to the other but, rather, decreases or slows the ability of the protein to do so. For example, in some embodiments, the mutation(s) slows the efficiency of the hydrolysis of GTP to GDP, thereby rendering the protein into a predominantly active conformational state. In some embodiments, the mutation(s) slows the hydrolysis of GTP by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, in comparison to the hydrolysis of GTP in the wild type Ras protein. In another embodiment, the mutation(s) slows the efficiency of the binding of GTP into the active site of the Ras protein. In some embodiments, the mutation(s) slows the efficiency of the binding of GTP into the active site of the Ras protein by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, in comparison to the binding of GTP into the active site of the wild type Ras protein. In other embodiments, the mutation(s) inhibits the removal of GDP from the binding pocket of Ras, thereby rendering the Ras protein into a predominantly inactive state. In some embodiments, the mutation(s) inhibit or slow the exchange of GDP for GTP by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, in comparison to the exchange of these nucleotides in the wild type Ras protein.

Methods for engineering a mutation or substitution into the primary amino acid sequence of a Ras protein are well known in the art via standard techniques. In certain embodiments, the mutant Ras protein for use in the present methods and compositions includes one or more conservative substitutions. In certain embodiments, the mutant Ras protein for use in the present methods and compositions includes one or more non-conservative substitutions or substantial substitutions. Substantial modifications in the biological properties of Ras proteins are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Non-conservative substitutions will entail exchanging a member of one of these classes for another class. In certain embodiments, amino acids may be grouped according to common side-chain properties: (1) hydrophobic: Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe; (7) large hydrophobic: Met, Val, Leu, Ile.

In further embodiments, the mutant Ras proteins for use in the methods disclosed herein may comprise one or more non-naturally occurring or modified amino acids. A “non-naturally occurring amino acid residue” refers to a residue, other than those naturally occurring amino acid residues, which is able to covalently bind adjacent amino acid residues(s) in a polypeptide chain. Non-natural amino acids include, but are not limited to, homo-lysine, homo-arginine, homo-serine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, citrulline, pentylglycine, pipecolic acid and thioproline. Modified amino acids include natural and non-natural amino acids which are chemically blocked, reversibly or irreversibly, or modified on their N-terminal amino group or their side chain groups, as for example, N-methylated D and L amino acids, side chain functional groups that are chemically modified to another functional group. For example, modified amino acids include methionine sulfoxide; methionine sulfone; aspartic acid-(beta-methyl ester), a modified amino acid of aspartic acid; N-ethylglycine, a modified amino acid of glycine; or alanine carboxamide and a modified amino acid of alanine. Additional non-natural and modified amino acids, and methods of incorporating them into proteins and peptides, are known in the art. Sandberg et al., (1998) J. Med. Chem. 41: 2481-91. Xie et al., (2005) Curr. Opin. Chem. Biol. 9: 548-554. Hodgson et al., (2004) Chem. Soc. Rev. 33: 422-430.

In some embodiments, the mutant Ras proteins for use in the methods described herein can be isolated from cells (such as a cancer cell) by an appropriate purification scheme using standard protein purification techniques. In another embodiment, the mutant Ras proteins for use in the present methods are produced by recombinant DNA techniques. Alternative to recombinant expression, the mutant Ras proteins for use in the present methods can be synthesized chemically using standard peptide synthesis techniques.

In some embodiments of any of the embodiments provided herein, the Ras protein and/or SUMO protein comprises an affinity tag. Non-limiting examples of affinity tags include histidine (His) tags, V5 tags, FLAG tags, influenza hemagglutinin (HA) tags, Myc tags, VSV-G tags, and thioredoxin (Trx) tags.

The present disclosure is based, in part, on the discovery of the fact that all three isoforms of RAS proteins were subjected to modification by SUMO3. Lys42, a conserved residue, was the primary site of sumoylation. Moreover, PIASγ an E3 ligase, specifically interacted with RAS and promoted its SUMO E3 ligase and/or sumoylation. Significantly, expression of oncogenic RASV12 is associated with increased sumoylation whereas expression of RASN17 was correlated with decreased sumoylation, as well as monoubiquitination. A series of biochemical and molecular characterization revealed that sumoylation was essential for activating RAS proteins and its downstream signaling. Furthermore, sumoylation is required for the sustained activation of RAS proteins and its downstream signaling. Significantly, SUMO-resistant mutant of KRAS greatly reduced cellular migration and invasiveness in vitro and suppressed tumor development in xenograft mice. Combined, these results strongly suggest that sumoylation is an undocumented post-translational mechanism that controls RAS activities in vivo. RAS proteins are regulated by post-translotional modifications include farnesylation, carboxylmethylation, and palmitoylation (Ref 1, 7, 8, 9, 10). Nascent RAS proteins are first processed by three-step modifications that lead to generation of lipidated proteins with hydrophobic C-termini that mediate association with cellular membranes 1. RAS proteins are also modified by palmitoylation through covalent attachment of 16-carbon palmitoyl chain to cysteine residues. Palmitoylation is a necessary step for translocation of RAS proteins from the endomembrane system to the cell surface membrane (Ref 1, 7). Unlike farnesylation, palmitoylation is reversible.

Extensive research in the past has also revealed that RAS proteins are subjected to other posttranslational modifications including phosphorylation, ubiquitination, acetylation, and S-nitrosylation (Ref 8, 11, 12, 13, 14, 15). For example, K-RAS4B is phosphorylated on serine 181 by protein kinase C and the phosphorylation is involved in the negative regulation of its association with the plasma membrane. RAS proteins are also modified by monoubiquitination and bi-ubiquitination (Ref. 11, 12, 13). Lys117, Lys147 and Lys170 are likely the sites of ubiquitination (Ref. 1). The E3 ligase specific for RAS ubiquitination is RABEX5 (Ref 13). Cys118, a highly conserved site in RAS isoforms and orthologs, can be modified by nitrosylation (Ref. 16). S-nitrosylation facilitates guanine nucleotide exchange, promoting efficient RAS activation (Ref 16, 17).

The SUMO (small ubiquitin-related modifier) pathway resembles the ubiquitin pathway. It consists of three enzymatic components including the E 1 activating enzyme (SAE1/2), the E2 conjugating enzyme (UBC9) and a series of E3 ligases that promote sumoylation in a substrate-specific manner (Ref. 18, 19). Sumoylation occurs on those lysine residues immediately after amino acids of a hydrophobic nature. Mammalian cells have three structural homologs: SUMO1, SUMO2, and SUMO3. Similar to other types of post-translational modifications, sumoylation is reversed by the proteolytic cleavage of isopeptidases of the SENP family (Ref 19). Extensive research has uncovered important functions of sumoylation that control the subcellular localization, stability and enzyme activities of target proteins. Sumoylation is also critical in development and cell biology, as its disruption either causes abnormal cellular growth and differentiation or leads to embryonic lethality (Ref 20).

The present disclosure provides a method for treating cancer in a mammalian subject, comprising administering to the subject an effective amount of an agent that interferes with, or decreases, sumoylation of a Ras protein. In certain embodiments, the agent decreases expression and/or conjugation of at least one member of the SUMO conjugation pathway. In certain embodiments, the agent is capable of decreasing the amount of SUMO conjugation to a Ras protein. The reference may include a control cell, such as a non-cancer cell of the same type as the cancer cell or a standard based on such a control cell, as described herein.

Reducing the level of expression of at least one member of the SUMO conjugation pathway in a cancer cell may reduce cancer cell proliferation, migration and/or survival by at least about 5%, at least about 10%, at least about 20%, at least about 30%, at least about 40%, at least about 50%, at least about 60%, at least about 70%, at least about 80%, at least about 85%, or at least about 90%. A reduction in cancer cell proliferation, migration and/or survival may be achieved by reducing sumoylation of a Ras protein by about 90%, about 80%, about 70%, about 60%, about 50%, about 40%, about 30%, about 20%, about 10%, about 5% or less as compared to the level of the sumoylated Ras prior to treatment with the agent.

The present disclosure provides a method of predicting the risk of progression of a cancer to a more aggressive form of cancer, comprising detecting the level of sumoylated Ras compared to a control cell. In certain embodiments, an increased level of sumoylated Ras in a cancer cell relative to a control cell may identify the cancer cell as being at risk for progressing to a more aggressive form. In certain embodiments, an unchanged or similar level of sumoylated Ras in a cancer cell relative to the level of sumoylated Ras in a control or reference may identify the cancer cell as being at risk for progressing to a more aggressive form.

A control or reference cell may be a non-cancerous cell, or a cancer cell known to be non-aggressive, as described above.

Also encompassed by the present disclosure is a method to predict a subject's cancer's responsiveness to a therapy by determining level of sumoylated Ras compared to a control cell.

The present agent/composition may produce cellular effects including, but not limited to, one or more of the following: increased apoptosis of cells, increased cell death, decreased or inhibited cell growth, inhibiting DNA synthesis in the cell, reduced tumor volume, reduced tumor burden, clearance of a tumor, inhibition of tumor growth or tumor cell proliferation, inhibition of metastases, reduced metastases and enhanced survival of a subject bearing tumor or cancer cells having increased level of sumoylated Ras.

The present disclosure provides a method of screening for cancer in a subject comprising detecting the level of sumoylated Ras in a sample from the subject and comparing the level of sumoylated Ras to the level of sumoylated Ras in a control, where an increased level of sumoylated Ras identifies the subject as at risk of having cancer or as having cancer.

The present disclosure also provides a method of assessing an agent for chemotherapeutic potential, comprising contacting a cell with the agent and evaluating the level of sumoylated Ras in the contacted cell, where a decrease in the level of sumoylated Ras in the contacted cell relative to an untreated cell identifies the chemotherapeutic potential of the agent. A control or reference may be the level of sumoylated Ras in a control or reference cell, such as a non-cancerous cell or a cancer cell with known responsiveness to a therapeutic. Alternatively, a standard value developed by analyzing the results of a population of non-cancer cells or cancer cells with known responsivities to a therapeutic may be used.

### Sumoylation Inhibitors

The present agents include a sumoylation inhibitor. The term “sumoylation inhibitor” or “SUMO inhibitor” as used herein refers to any agent that binds one or more subunit of a sumoylation enzyme, thereby inhibiting the conjugation of a SUMO protein to a target protein, e.g., a Ras protein. Such inhibitors may also inhibit one or more SUMOylation enzymes. The term “SUMOylation enzyme” or “SUMO enzyme” as used herein refers to SUMO activation enzyme SUMO E1, SUMO conjugating enzyme SUMO E2 or any one or more of SUMO E3 ligases.

In some embodiments, the SUMO inhibitors are SUMO E1 inhibitors. The term “SUMO E1” as used herein refers to SUMO activating enzyme SUMO E1, which is made up of subunits SAE1 and SAE2/Uba2. In certain embodiments, the SUMO E1 inhibitor inhibits both E1 subunits, SAE1 and SAE2. In these embodiments, the inhibitors may inhibit one subunit to a greater degree than the other, or they may inhibit the two subunits equally. In other embodiments, the inhibitors inhibit one subunit only.

The term “SUMO E2” as used herein refers to SUMO conjugating enzyme SUMO E2, which is made up of a single subunit, Ubc9. In certain embodiments, the SUMO E2 inhibitor inhibits Ubc9 only, or the inhibitor may inhibit Ubc9 and one or more SUMO E1 subunits. In these embodiments, the inhibitors may inhibit Ubc9 to a greater degree than the one or more E1 subunits, or they may inhibit two or more of the subunits equally.

In certain embodiments, the agent is an inhibitor of at least one member of the SUMO conjugation pathway. In certain embodiments, a member of the SUMO conjugation pathway includes but is not limited to proteins that can conjugate and/or can be conjugated to at least one of SUMO1, SUMO2, SUMO3, and SUMO4 proteins. In some embodiments, an agent capable of decreasing the level of expression of a component of the SUMO conjugation pathway may include an agent that decreases SUMO mRNA or protein levels in the cell contacted with the agent.

In some embodiments, the agent that decreases the level of expression and/or conjugation of at least one member of the SUMO conjugation pathway may include a gene expression repressor. The agent that decreases the level of expression and/or conjugation of at least one member of the SUMO conjugation pathway may also include agents that bind to the member of the SUMO conjugation pathway directly or indirectly and decrease the effective level or activity of the member, for example, by inhibiting the binding or other activity of SUMO1, SUMO2, SUMO3 or SUMO4. The agent capable of decreasing the level of expression and/or conjugation of at least one member of the SUMO conjugation pathway may also include agents that decrease free SUMO1, SUMO2 SUMO3 or SUMO4 levels or increase free SUMO1, SUMO2, SUMO3 or SUMO4 levels.

In certain embodiments, a member of the SUMO conjugation pathway includes proteins and enzymes that are specific to the SUMO conjugating or de-conjugating processes. In the embodiments described herein, a member of the SUMO conjugation pathway includes SUMO 1, SUMO2 SUMO3 or SUMO4, activating enzymes (SUMO E1), the conjugating enzyme Ubc9 (SUMO E2), ligating enzymes (SUMO E3; Ye, J. Biol. Chem. 284: 8223-8227 (2009)), and/or deconjugating enzymes (e.g., SENPs; Xu et al., Antioxid. Redox Signal. 11: 1453-1484 (2009)). Other members of the SUMO conjugation pathway include RWD-containing sumoylation enhancer (RSUME) (Carbia-Nagashima et al., Cell 131: 309-323 (2007)) and SUMO E3 ligase PIASγ (Wang & Baneijee, Oncol. Rep. 11: 1319-1324 (2004)).

The agents for use in the present methods and compositions can be any of a small molecule compound, an antibody, an antibody fragment, a polypeptide, a carbohydrate, an inhibitory nucleic acid, or any combination thereof.

In certain embodiments, the agents for use in the present methods and compositions are antibodies or a fragment (or fragments) of an antibody, that bind specifically to a sumoylated Ras protein. In certain embodiments, the agents for use in the present methods and compositions are antibodies or a fragment (or fragments) of an antibody, that bind specifically to an epitope that includes at least one sumoylation site (such as amino acid residue 42, and amino acid residue 104) of a Ras protein.

The antibodies can be full-length or can include a fragment (or fragments) of the antibody, e.g., having an antigen-binding portion. Fragments of the antibody include, but are not limited to, Fab, F(ab′)2, Fab′, F(ab)′, Fv, single chain Fv (scFv), bivalent scFv (bi-scFv), trivalent scFv (tri-scFv), Fd, dAb fragment (e.g., Ward et al., Nature, 341:544-546 (1989)), an isolated CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present disclosure. Bird et al. Science, 1988, 242:423-426. Huston et al., Proc. Natl. Acad. Sci. USA, 1988, 85:5879-5883.

The antibodies of the present disclosure may be generated by any suitable method known in the art.

Polyclonal antibodies to a Ras protein (e.g., a sumoylated Ras protein) or a fragment thereof (e.g., a fragment of a Ras protein sumoylated or non-sumoylated at amino acid residue 42) can be produced by various procedures well known in the art. In certain embodiments, a Ras protein (e.g., a sumoylated Ras protein) or a fragment thereof (e.g., a fragment of a Ras protein sumoylated or non-sumoylated at amino acid residue 42) is administered to a host animal to induce the production of sera containing polyclonal antibodies specific for the Ras protein (e.g., a sumoylated Ras protein), or a fragment thereof (e.g., an epitope comprising amino acid residue 42 of a Ras protein where amino acid residue 42 may be sumoylated or non-sumoylated). The immunized animal can be any animal that is capable of producing recoverable antibodies when administered an immunogen, such as, but not limited to, rabbits, mice, rats, hamsters, goats, horses, monkeys, baboons and humans. In one aspect, the host is transgenic and produces human antibodies, e.g., a mouse expressing the human immunoglobulin gene segments. U.S. Pat. Nos. 8,236,311; 7,625,559 and 5,770,429, the disclosure of each of which is incorporated herein by reference in its entirety. Lonberg et al., Nature 368(6474): 856-859, 1994. Lonberg, N., Handbook of Experimental Pharmacology 113:49-101, 1994. Lonberg, N. and Huszar, D., Intern. Rev. Immunol., 13: 65-93, 1995. Harding, F. and Lonberg, N., Ann. N.Y. Acad. Sci., 764:536-546, 1995.

Various adjuvants may be used to increase the immunological response, depending on the host species, and include but are not limited to, Freund's (complete and incomplete), mineral gels such as aluminum hydroxide, surface active substances such as lysolecithin, pluronic polyols5 polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. In certain embodiments, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammerling, et al., in: Monoclonal Antibodies and T-Cell Hybridomas 563-681 (Elsevier, N.Y., 1981) (said references incorporated by reference in their entireties). Methods for producing and screening for specific antibodies using hybridoma technology are routine and well known in the art.

In certain embodiments, the present disclosure provides for a method for making a hybridoma that expresses an antibody that specifically binds to a Ras protein (e.g., a sumoylated Ras protein), a fragment thereof (e.g., an epitope comprising amino acid residue 42 of a Ras protein where amino acid residue 42 may be sumoylated or non-sumoylated). The method contains the following steps: immunizing an animal with a composition that includes a Ras protein (e.g., a sumoylated Ras protein), or a fragment thereof (e.g., an epitope comprising amino acid residue 42 of a Ras protein where amino acid residue 42 may be sumoylated or non-sumoylated); isolating splenocytes from the animal; generating hybridomas from the splenocytes; and selecting a hybridoma that produces an antibody that specifically binds to a Ras protein (e.g., a sumoylated Ras protein), a fragment thereof (e.g., an epitope comprising amino acid residue 42 of a Ras protein where amino acid residue 42 may be sumoylated or non-sumoylated). Kohler and Milstein, Nature, 256: 495, 1975. Harlow, E. and Lane, D. Antibodies: A Laboratory Manual, Cold Spring Harbor Laboratory Press, Cold Spring Harbor, N.Y., 1988.

Adjuvants that may be used to increase the immunogenicity of one or more of the antigens, include any compound or compounds that act to increase an immune response to peptides or combination of peptides. Non-limiting examples of adjuvants include alum, aluminum phosphate, aluminum hydroxide, MF59 (4.3% w/v squalene, 0.5% w/v polysorbate 80 (Tween 80), 0.5% w/v sorbitan trioleate (Span 85)), CpG-containing nucleic acid, QS21 (saponin adjuvant), MPL (Monophosphoryl Lipid A), 3DMPL (3-O-deacylated MPL), extracts from Aquilla, ISCOMS (see, e.g., Sjolander et al. (1998) J. Leukocyte Biol. 64:713; WO90/03184; WO96/11711; WO 00/48630; WO98/36772; WO00/41720; WO06/134423 and WO07/026190), LT/CT mutants, poly(D,L-lactide-co-glycolide) (PLG) microparticles, Quil A, interleukins, Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The methods for making a “humanized antibody” are well-known in the art. This is a technique of reconstructing the antigen binding site of a non-human antibody (e.g. a rodent antibody) on a human antibody (Jones, P. T. et al., Nature 321, 5225-525 (1986); Verhoeyen, M. et al., Science 239, 1534-1536 (1988); Riechmann, L. et al., Nature 332,323-327 (1988)). In general, the humanized antibody or fragment thereof comprises substantially at least one, and more preferably two, variable domains (Fab, Fab′, F(ab′)2, Fabc, Fv) in which all or substantially all of the complementarity determining regions (CDR) correspond to those of a non-human immunoglobulin, and all or substantially all of the framework regions (FR) are those of a human immunoglobulin consensus sequence. The humanized antibody optimally also comprises at least a portion of an immunoglobulin constant region (Fc), typically that of a human immunoglobulin. Ordinarily, the antibody contains both the light chain as well as at least the variable domain of a heavy chain. The antibody also may include the CH1, hinge, CH2, CH3, and/or CH4 regions of the heavy chain. The humanized antibody may be selected from any class of immunoglobulins, including IgM, IgG, IgD, IgA and IgE, and any isotype, including IgG 1, IgG2, IgG3 and IgG4. The humanized antibody may comprise sequences from more than one class or isotype, and selecting particular constant domains to optimize desired effector functions is within the ordinary skill in the art. The FR and CDR regions of the humanized antibody need not correspond precisely to the parental sequences, e.g., the import CDR or the consensus FR may be mutagenized by substitution, insertion or deletion of one or more residues so that the CDR or FR residue at that site does not correspond to either the consensus or the import antibody. Such mutations, however, will not be extensive and will not dramatically affect binding of the antibody to the binding target. The term “humanized antibody” further includes antibodies and polypeptides rendered non-immunogenic, or having reduced immunogenicity relative to the native antibody, to a human by the method of determining at least part of the amino acid sequence of the antibody or polypeptide (preferably that part of non-human origin such as a VH or VK region of a non-human antibody), identifying in the amino acid sequence one or more potential epitopes for human T-cells, and modifying the amino acid sequence(s) of the one or more epitopes to eliminate at least one of the T-cell epitopes identified in order to eliminate or reduce the immunogenicity of the protein or portions thereof when exposed to the human immune system.

After the host is immunized and the antibodies are produced, the antibodies are assayed to confirm that they are specific for the antigen of interest and to determine whether they exhibit any cross reactivity with other antigens. One method of conducting such assays is a sera screen assay. U.S. Patent Publication No. 2004/0126829. Antibodies specific for a Ras protein (e.g., a sumoylated Ras protein), or a fragment thereof (e.g., an epitope comprising amino acid residue 42 of a Ras protein where amino acid residue 42 may be sumoylated or non-sumoylated) can be characterized for binding to the antigen or epitope by a variety of known techniques. In certain embodiments, in an ELISA, microtiter plates are coated with the Ras protein (e.g., a sumoylated Ras protein), or a fragment thereof (e.g., an epitope comprising amino acid residue 42 of a Ras protein where amino acid residue 42 may be sumoylated or non-sumoylated) in PBS, and then blocked with irrelevant proteins such as bovine serum albumin (BSA) diluted in PBS. Dilutions of plasma from mice immunized by the Ras protein (e.g., a sumoylated Ras protein), or a fragment thereof are added to each well and incubated. The plates are washed and then incubated with a secondary antibody conjugated to an enzyme (e.g., alkaline phosphatase). After washing, the plates are developed with the enzyme's substrate (e.g., ABTS), and analyzed at a specific OD. In certain embodiments, to determine if the selected monoclonal antibodies bind to unique epitopes, the antibody can be biotinylated which can then be detected with a streptavidin labeled probe. In certain embodiments, antibodies specific for a Ras protein (e.g., a sumoylated Ras protein), or a fragment thereof (e.g., an epitope comprising amino acid residue 42 of a Ras protein where amino acid residue 42 may be sumoylated or non-sumoylated) can be tested for reactivity with the Ras protein, or a fragment thereof, by Western blotting.

Hybridomas that produce antibodies that bind, preferably with high affinity, to the Ras protein (e.g., a sumoylated Ras protein), or a fragment thereof (e.g., an epitope comprising amino acid residue 42 of a Ras protein where amino acid residue 42 may be sumoylated or non-sumoylated) can than be subcloned and further characterized. One clone from each hybridoma, which retains the reactivity of the parent cells (by ELISA), can then be chosen for making a cell bank, and for antibody purification.

To purify the antibodies specific for a Ras protein (e.g., a sumoylated Ras protein), or a fragment thereof (e.g., an epitope comprising amino acid residue 42 of a Ras protein where amino acid residue 42 may be sumoylated or non-sumoylated), supernatants from the cultured hybridomas can be filtered and concentrated before affinity chromatography with protein A-Sepharose (Pharmacia, Piscataway, N.J.).

Antibodies, or antigen-binding fragments, variants or derivatives thereof of the present disclosure can also be described or specified in terms of their binding affinity to an antigen. The affinity of an antibody for an antigen can be determined experimentally using any suitable method (see, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer.

Antibody fragments may be generated by traditional means, such as enzymatic digestion, or by recombinant techniques. Hudson et al. Nat. Med. 9:129-134, 2003. Various techniques have been developed for the production of antibody fragments. Traditionally, these fragments were derived via proteolytic digestion of intact antibodies (see, e.g., Morimoto et al., J. Biochem. Biophys. Methods 24:107-17, 1992; and Brennan et al., Science 229:81-3, 1985). However, these fragments can now be produced directly by recombinant host cells. Fab, Fv, and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments (Carter et al., Bio/Technology 10:163-7, 1992). In another approach, F(ab′)₂ fragments are isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner.

In certain embodiments, the present antibody or a fragment thereof may comprise at least one constant domain, such as, (a) an IgG constant domain; (b) an IgA constant domain, etc.

All antibody isotypes are encompassed by the present disclosure, including IgG (e.g., IgG1, IgG2, IgG3, IgG4), IgM, IgA (IgA1, IgA2), IgD or IgE. The antibodies or antigen-binding portions thereof may be mammalian (e.g., mouse, human) antibodies or a fragment thereof. The light chains of the antibody may be of kappa or lambda type.

The antibodies or a fragment thereof of the present disclosure may be monospecific, bi-specific or multi-specific. Multi-specific or bi-specific antibodies or fragments thereof may be specific for different epitopes of one target polypeptide (e.g., sumoylated Ras) or may contain antigen-binding domains specific for more than one target polypeptide (e.g., antigen-binding domains specific for sumoylated Ras and other antigen relating to cancer). In one embodiment, a multispecific antibody or antigen-binding portion thereof comprises at least two different variable domains, wherein each variable domain is capable of specifically binding to a separate antigen or to a different epitope on the same antigen. Tutt et al., 1991, J. Immunol. 147:60-69. Kufer et al., 2004, Trends Biotechnol. 22:238-244. The present antibodies can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present disclosure includes bi-specific antibodies wherein one arm of an immunoglobulin is specific for sumoylated Ras, and the other arm of the immunoglobulin is specific for a second therapeutic target or is conjugated to a therapeutic moiety.

The antibodies may be chimeric, humanized, single chain, or bi-specific. All antibody isotypes are encompassed by the present disclosure, including, IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

Any of the various types of antibodies can be used for this purpose, including, but not limited to, polyclonal antibodies, monoclonal antibodies, humanized antibodies, human antibodies (e.g., generated using transgenic mice, etc.), single chain antibodies (e.g., single chain Fv (scFv) antibodies), heavy chain antibodies and chimeric antibodies. The antibodies can be from various species, such as rabbits, mice, rats, goats, chickens, guinea pigs, hamsters, horses, sheep, llamas etc.

In certain embodiments, the inhibitor of the member of the SUMO conjugation pathway is an inhibitory RNA. An inhibitory RNA may be a sequence complementary to a portion of an RNA sequence encoding SUMO. In embodiments, an inhibitory RNA may refer to, but is not limited to dsRNA, siRNA, shRNA, miRNA, piRNA, RNAse external guide sequences, ribozymes, and other short catalytic RNAs. In embodiments, the inhibitory RNA comprises a miRNA comprising a sequence complementary to a sequence of Ubc9, SUMO1, SUMO2, SUMO3, SUMO4, or any combination thereof. In embodiments, the amino acid sequences or the RNA sequences encoding the member of the SUMO conjugation pathway, i.e., SUMO1-4, SUMO activating, conjugating and ligating enzymes, are selected from any of GenBank Accession Nos. or NCBI Reference Sequence Nos. AAC50996.1, NM_001005781.1, NM_001005782.1, NP_003343.1, NM_03352.4 (SUMO1); NP_008868.3, NM_001005849.1, NM_006937.3 (SUMO2); NP_008867.2, NP_001273345.1, NM_0069362 (SUMO3); NP_001002255.1 (SUMO4); AF002385.1 (Ubc9); NM_006099 (SUMO ligating enzyme PIAS3); NM_173206.2, NM_004671.2 (SUMO ligating enzyme PIAS2); NM_01.5897.2 (SUMO ligating enzyme PIAS4); AF077952.1 (SUMO ligating enzyme PIASγ); NM_001145713.1, NM_005.500.2, NM_001145714.1, NM_003334.3, NM_153280.2, NM_005499.2, NR_027280.1, AFI 109.56.1, AF110957.1, AF079566.1, AF090384.1, AF090385.2, AF046025.2, BC003611.2, BC000344.2, BC018271.1, BC003153.1, BT009781.1, BT007290.1, CR456756.1, and AK315624.1. Methods of synthesis, design and delivery of inhibitory RNA are well known in the art, see, e.g., Integrated DNA Technologies, Inc. “Dicer Substrate RNAi Design” at the idtdna.com site; Krebs and Alsberg, Chem. Eur. J. 17: 3054-3062 (2011); Lin et al., RNA 17:603-612 (2011).

In certain embodiments, the agent is an activator of SUMO deconjugation. In certain embodiments, the agent is an activator of a sentrin/SUMO-specific protease (SENP). Non-limiting examples of SENPs include SENP1, SENP, SENP3, SENP, SENP, SENP6, and SENP7. Xu et al., Antioxid. Redox Signal. 11: 1453-1484 (2009).

### Determining Sumoylation Level of Ras

In certain embodiments, the present methods or compositions may decrease the sumoylation of a Ras protein by decreasing the level of expression of the member of the SUMO conjugation pathway, including, e.g., decreasing the RNA levels of SUMO1, SUMO2, SUMO3 or SUMO4, decreasing the amount of conjugated SUMO1, SUMO2, SUMO3 or SUMO4, decreasing the amount of free SUMO1, SUMO2, SUMO3 or SUMO4, or a combination thereof. In embodiments, SUMO is at least one of SUMO1, SUMO2, SUMO3, and SUMO4.

The level of sumoylated Ras in the cell may be evaluated by a variety of techniques, as will be appreciated by one of skill in the art. For example, the level of sumoylated Ras may be evaluated by detecting the presence and/or quantifying the amount of protein or mRNA using techniques including, but not limited to. Western blot, ELISA, Northern blot, real time PCR, immunofluorescence, or FACS analysis. The expression of sumoylated Ras may be evaluated by immunofluorescence by visualizing cells stained with a fluorescently-labeled SUMO-specific antibody, Western blot analysis of SUMO protein expression, and RT-PCR of SUMO transcripts. The expression of the SUMO conjugation pathway may be evaluated by microarray analysis.

In certain embodiments, SUMO conjugation can be determined by measuring mRNA or protein levels of at least one member of the SUMO conjugation pathway. SUMO conjugation can be determined by measuring the activity of SUMO conjugation of a target protein, e.g., a Ras protein. The activity of SUMO conjugation can be determined by isolating the components of the SUMO conjugation pathway from tissue samples, adding SUMO1, SUMO2, SUMO3 or SUMO4, ATP, an ATP regenerating system, and a SUMO conjugation target protein (e.g., a Ras protein), and analyzing the results of the reaction by Western blotting using an antibody directed against the target protein (e.g., a Ras protein). The activity of SUMO conjugation of the target protein can then be identified by the appearance of higher molecular weight bands that disappear after incubation with SENP. SUMO deconjugation can be measured using extracts from tissue containing tissue SENPs, adding a SUMO conjugated protein such as polySUMO and measuring deconjugation by Western blotting (polySUMO deconjugated to monoSUMO), similar to the analysis for conjugation.

The level of sumoylated Ras may be compared to a control or reference. A control or reference may include, for example, the level of expression of the member of the SUMO conjugation pathway in a control cell or reference cell, such as a non-cancerous cell, optionally from a similar type, or the same type, of tissue as the sample. A control may include an average range of the level of sumoylated Ras from a population of non-cancer cells, or alternatively, a standard value developed by analyzing the results of a population of non-cancer cells. A control or reference may include, for example, the level of sumoylated Ras in an untreated sample of tissue of similar type, or the same type as the sample contacted with an agent. Those skilled in the art will appreciate that a variety of controls or references may be used.

### Downstream Ras Effectors

The present disclosure provides methods of modulating sumoylation of Ras in cancer cells to decrease the aberrant signaling of downstream Ras effector molecules.

The present agent or composition may have one or more of the following effects on cancer cells or the subject: cell death; decreased cell proliferation; decreased numbers of cells; inhibition of cell growth; apoptosis; necrosis; mitotic catastrophe; cell cycle arrest; decreased cell size; decreased cell division; decreased cell survival; decreased cell metabolism; markers of cell damage or cytotoxicity; indirect indicators of cell damage or cytotoxicity such as tumor shrinkage; improved survival of a subject; or disappearance of markers associated with undesirable, unwanted, or aberrant cell proliferation. U.S. Patent Publication No. 20080275057.

In certain embodiments, the one or more effector proteins are members of Ras-regulated signaling pathways that control one or more of actin cytoskeletal integrity, cell growth and/or proliferation, cell differentiation, cell adhesion, apoptosis, and/or cell migration.

In certain embodiments, the one or more effector proteins are members of a mitogen activated protein kinase (MAPK) signaling pathway, including, but not limited to, the extracellular signal regulated mitogen-activated protein kinase (ERK-MAPK) signaling pathway. The MAPK signaling pathway is a main component in several steps of tumorigenesis including cancer cell proliferation, migration, invasion and survival. Overall, the activation of a MAPK employs a core three-kinase cascade. The extracellular mitogen binds to the membrane receptor (e.g., receptor tyrosine kinases, cytokine receptors, and some G protein-coupled receptors), which allows Ras (a GTPase) to swap its GDP for a GTP. It can now activate a MAPK kinase kinase (MAP3K or MAPKKK; e.g., Raf), which phosphorylates and activates a MAPK kinase (MAP2K, MEK, or MKK), which then phosphorylates and activates a MAPK (e.g., ERKs). Upon activation, MAPKs can phosphorylate and activate a variety of intracellular targets including transcription factors, nuclear pore proteins, membrane transporters, cytoskeletal elements, and other protein kinases. The extracellular signal-regulated kinase (ERK) pathway (also referred to as the ERK-MAPK pathway, or the p44/42 MAPK pathway) is activated by a wide variety of mitogenic stimuli that interact with structurally distinct receptors and thus represents a convergence point for most, if not all, mitogenic signaling pathways (Seger R. et al., FASEB J., 1995, 9: 726-735; Lewis T. S. et al., Adv. Cancer Res., 1998, 74: 49-139; and Pearson G. et al., Endocr. Rev., 2001, 22: 153-183). Any component of the MAPK signaling pathway or the ERK pathway may be the effector proteins, including, but not limited to, RAF, MEK, MAPK (ERK), or combinations thereof. Any isoform of any component the MAPK pathway may be the effector proteins, including, but not limited to, BRAF, CRAF, ARAF; MEK1, MEK2, MKK3, MKK4, MKKS, MKK6, or MKK7; ERK1, ERK2, p38, JNK, ERKS, or combinations thereof.

Non-limiting examples of downstream Ras effectors include, Abl, Aurora-A, Aurora-B, Aurora-C, ATK, bcr-Abl, Blk, Brk, Btk, c-Kit, c-Met, c-Src, CDK1, CDK2, CDK4, CDK6, cRafl, CSFIR, CSK, EGFR, ErbB2, ErbB3, ErbB4, ERK, Fak, fes, FGFR1, FGFR2, FGFR3, FGFR4, FGFR5, Fgr, FLK-4, Flt-1, Fms, Fps, Frk, Fyn, Hck, IGF-1R, INS-R, Jak, KDR, Lck, Lyn, MEK, p38, PDGFR, PIK, PKC, PYK2, Ros, Tie1, Tie2, Trk, Yes, Zap70, and combinations thereof. Magnuson et al., Seminars in Cancer Biology, 5:247-252 (1994).

Non-limiting examples of downstream Ras effectors also include, PI3K, AKT, CREB, p27, FOXO, PtdIns-3ps, mTOR, p70, 4EBP1, and combinations thereof.

The present method or composition may reduce MAPK signaling, reduce phosphorylation of components of the MAPK signaling pathways (e.g., phosphorylated MEK, phosphorylated ERK, etc.), reduce levels of activated components of the MAPK signaling pathways (e.g., including but not limited to members of the Ras/Raf/MEK/ERK pathways), and/or sequester components of the MAPK signaling pathways and prevent signaling. Brunet A. et al., EMBO J 1999, 18: 664-674.

### Conditions to be Treated and Pharmaceutical Compositions

Cells such as, for example, one or more cancer cells, may be contacted with an agent directly or indirectly in vivo, in vitro, or ex vivo. In certain embodiments, the cancer cell is associated with an increased level of at least one sumoylated Ras protein relative to a control cell. Contacting encompasses administration to a cell, tissue, mammal, patient, or human. Contacting a cell also includes adding an agent to a cell culture. Other suitable methods may include introducing or administering an agent to a cell, tissue, mammal, or patient using appropriate procedures and routes of administration. In an aspect the disclosure provides a method of reducing proliferation of a cancer cell comprising contacting the cancel cell with an agent that decreases the levels or amounts of sumoylated Ras protein(s) so as to reduce proliferation and/or migration of the cancer cell. In an aspect the disclosure provides a method of reducing proliferation or promoting differentiation of a cancer cell comprising contacting the cancel cell with an agent that decreases the levels or amounts of sumoylated Ras protein(s) so as to increase differentiation of the cancer cell. In embodiments, the method comprises contacting the cell with an agent to decrease the level of at least one sumoylated Ras protein so as to reduce proliferation and/or migration, or increase differentiation of the cancer cell.

The present disclosure provides for a method of treating a disease such as cancer in a subject comprising the step of administering the present agent or composition to the subject. The present disclosure also provides for methods of decreasing or inhibiting proliferation and/or migration of a cancer cell by contacting the cell with the present agent or composition. The present agent or composition interferes with sumoylation of a Ras protein by a SUMO protein. In certain embodiments, the present agent or composition interferes with sumoylation of a Ras protein at amino acid residue 42 and/or amino acid residue 104 by a SUMO protein.

The present disclosure also provides for a method of treating a disease comprising the step of administering to a patient a therapeutically effective amount of the present agent or composition.

The present disclosure also provides a method for inhibiting the growth of a cell in vitro, ex vivo or in vivo, where a cell, such as a cancer cell, is contacted with an effective amount of the present agent as described herein.

Pathological cells or tissue such as hyperproliferative cells or tissue may be treated by contacting the cells or tissue with an effective amount of the present agent/composition. The cells, such as cancer cells, can be primary cancer cells or can be cultured cells available from tissue banks such as the American Type Culture Collection (ATCC). The pathological cells can be cells of a systemic cancer, gliomas, meningiomas, pituitary adenomas, or a CNS metastasis from a systemic cancer, lung cancer, prostate cancer, breast cancer, hematopoietic cancer or ovarian cancer. The cells can be from a vertebrate, preferably a mammal, more preferably a human. U.S. Patent Publication No. 2004/0087651. Balassiano et al. (2002) Intern. J. Mol. Med. 10:785-788. Thorne, et al. (2004) Neuroscience 127:481-496. Fernandes, et al. (2005) Oncology Reports 13:943-947. Da Fonseca, et al. (2008) Surgical Neurology 70:259267. Da Fonseca, et al. (2008) Arch. Immunol. Ther. Exp. 56:267-276. Hashizume, et al. (2008) Neuroncology 10:112-120.

In vitro efficacy of the present composition can be determined using methods well known in the art. For example, the cytoxicity of the present agent may be studied by MTT [3-(4,5-dimethylthiazol-2-yl)-2,5-diphenyl tetrazolium bromide] cytotoxicity assay. MTT assay is based on the principle of uptake of MTT, a tetrazolium salt, by metabolically active cells where it is metabolized into a blue colored formazon product, which can be read spectrometrically. J. of Immunological Methods 65: 55 63, 1983. The cytoxicity of the present agent may be studied by colony formation assay. Functional assays for inhibition of VEGF secretion and IL-8 secretion may be performed via ELISA. Cell cycle block by the present agent may be studied by standard propidium iodide (PI) staining and flow cytometry. Invasion inhibition may be studied by Boyden chambers. In this assay a layer of reconstituted basement membrane, Matrigel, is coated onto chemotaxis filters and acts as a barrier to the migration of cells in the Boyden chambers. Only cells with invasive capacity can cross the Matrigel barrier. Other assays include, but are not limited to, cell viability assays, apoptosis assays, and morphological assays.

The present agent may be formulated into a pharmaceutical composition, where the present agent is present in amounts ranging from about 0.01% (w/w) to about 100% (w/w), from about 0.1% (w/w) to about 80% (w/w), from about 1% (w/w) to about 70% (w/w), from about 10% (w/w) to about 60% (w/w), or from about 0.1% (w/w) to about 20% (w/w).

The present compositions can be administered alone, or may be administered in combination with radiation, surgery, or another therapy (e.g., a chemotherapeutic agent, an angiogenesis inhibitor), to treat a disease such as cancer. Treatments may be sequential, with the present agent/composition being administered before or after the administration of other therapy. For example, the present agent or composition may be used to sensitize a cancer patient to radiation or chemotherapy. Alternatively, treatments may be administered concurrently.

Chemotherapeutic agents include, but are not limited to, a DNA alkylating agent, a topoisomerase inhibitor, an endoplasmic reticulum stress inducing agent, a platinum agent, an antimetabolite, a vincalkaloid, a taxane, an epothilone, an enzyme inhibitor, a receptor antagonist, a tyrosine kinase inhibitor, a boron radiosensitizer (i.e. velcade), and combinations thereof.

Examples of enzyme inhibitors include, but are not limited to farnesyltransferase inhibitors (Tipifarnib); CDK inhibitor (Alvocidib, Seliciclib); proteasome inhibitor (Bortezomib); phosphodiesterase inhibitor (Anagrelide; rolipram); IMP dehydrogenase inhibitor (Tiazofurine); and lipoxygenase inhibitor (Masoprocol). Examples of receptor antagonists include, but are not limited to ERA (Atrasentan); retinoid X receptor (Bexarotene); and a sex steroid (Testolactone).

Examples of tyrosine kinase inhibitors include, but are not limited to inhibitors to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib). A chemical equivalent of lapatinib is a small molecule or agent that is a tyrosine kinase inhibitor (TKI) or alternatively a HER-1 inhibitor or a HER-2 inhibitor. Several TKIs have been found to have effective antitumor activity and have been approved or are in clinical trials. Examples of such include, but are not limited to, Zactima (ZD6474), Iressa (gefitinib), imatinib mesylate (STI571; Gleevec), erlotinib (OSI-1774; Tarceva), canertinib (CI 1033), semaxinib (SU5416), vatalanib (PTK787/ZK222584), sorafenib (BAY 43-9006), sutent (SUI 1248) and lefltmomide (SU10l).

The present agent/composition may be co-administered with antiviral agents, anti-inflammatory agents or antibiotics. The agents may be administered concurrently or sequentially. The present agents can be administered before, during or after the administration of the other therapy.

Cancers that can be treated by the present agents include, but are not limited to, lung cancer; ear, nose and throat cancer; nervous system cancers; brain cancer; leukemia; colon cancer; melanoma; pancreatic cancer; mammary cancer; prostate cancer; breast cancer; hematopoietic cancer; ovarian cancer; basal cell carcinoma; biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; liver cancer; lymphoma including Hodgkin's and Non-Hodgkin's lymphoma; myeloma; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. U.S. Pat. No. 7,601,355.

The present composition may be administered by any method known in the art, including, without limitation, intranasal, oral, transdermal, ocular, intraperitoneal, inhalation, intravenous, ICV, intracisternal injection or infusion, subcutaneous, implant, vaginal, sublingual, urethral (e.g., urethral suppository), subcutaneous, intramuscular, intravenous, rectal, sub-lingual, mucosal, ophthalmic, spinal, intrathecal, intra-articular, intra-arterial, sub-arachinoid, bronchial and lymphatic administration. Topical formulation may be in the form of gel, ointment, cream, aerosol, etc; intranasal formulation can be delivered as a spray or in a drop; transdermal formulation may be administered via a transdermal patch or iontorphoresis; inhalation formulation can be delivered using a nebulizer or similar device. Compositions can also take the form of tablets, pills, capsules, semisolids, powders, sustained release formulations, solutions, suspensions, elixirs, aerosols, or any other appropriate compositions.

To prepare such pharmaceutical compositions, one or more of agent of the present disclosure may be mixed with a pharmaceutical acceptable carrier, adjuvant and/or excipient, according to conventional pharmaceutical agenting techniques. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers and adjuvants, see Remington's Pharmaceutical Sciences, edited by E. W. Martin (Mack Publishing Company, 18th ed., 1990). The compositions also can include stabilizers and preservatives.

Methods of determining the most effective means and dosage of administration can vary with the composition used for therapy, the purpose of the therapy, the target cell being treated, and the subject being treated. Treatment dosages generally may be titrated to optimize safety and efficacy. Single or multiple administrations can be carried out with the dose level and pattern being selected by the treating physician. Suitable dosage formulations and methods of administering the agents can be readily determined by those of skill in the art. For example, the composition is administered at about 0.01 mg/kg to about 200 mg/kg, about 0.1 mg/kg to about 100 mg/kg, or about 0.5 mg/kg to about 50 mg/kg. When the agents described herein are co-administered with another therapy, the effective amount may be less than when the agent is used alone.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer. If the composition is in the form of a gel, the composition may be rubbed onto a membrane of the patient, for example, the skin, preferably intact, clean, and dry skin, of the shoulder or upper arm and or the upper torso, and maintained thereon for a period of time sufficient for delivery of the present agent to the blood serum of the patient. The composition of the present disclosure in gel form may be contained in a tube, a sachet, or a metered pump. Such a tube or sachet may contain one unit dose, or more than one unit dose, of the composition. A metered pump may be capable of dispensing one metered dose of the composition.

This invention also provides the compositions as described above for intranasal administration. As such, the compositions can further comprise a permeation enhancer. Southall et al. Developments in Nasal Drug Delivery, 2000. The present agent may be administered intranasally in a liquid form such as a solution, an emulsion, a suspension, drops, or in a solid form such as a powder, gel, or ointment.

The composition containing the present agent can be administered by oral inhalation into the respiratory tract, i.e., the lungs.

The present agent may also be used alone or in combination with other chemotherapeutic agents via topical application for the treatment of localized cancers such as breast cancer or melanomas. The present agent may also be used in combination with narcotics or analgesics for transdermal delivery of pain medication.

This invention also provides the compositions as described above for ocular administration. As such, the compositions can further comprise a permeation enhancer. For ocular administration, the compositions described herein can be formulated as a solution, emulsion, suspension, etc. A variety of vehicles suitable for administering agents to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

The present agent can be given alone or in combination with other drugs for the treatment of the above diseases for a short or prolonged period of time. The present compositions can be administered to a mammal, preferably a human. Mammals include, but are not limited to, murines, rats, rabbit, simians, bovines, ovine, porcine, canines, feline, farm animals, sport animals, pets, equine, and primates.

The following are examples of the present invention and are not to be construed as limiting.

### EXAMPLES

### 6. EXAMPLE 1

### 6.1 Ras Proteins Are Modified by Sumoylation

Described herein is that sumoylation plays a major role in regulating stability, activity, and subcellular localization, we investigated whether RAS proteins were post-translationally modified by SUMOs. Applicant discovered that HRAS, KRAS and NRAS were all SUMO-modified and that SUMO3 is the most efficient modifier. Lys42 was the primary residue for sumoylation. Moreover, PIASγ is a specific E3 ligase for RAS sumoylation, and promoting RAS sumoylation in vitro. Significantly, expression of oncogenic RASV12 is associated with increased sumoylation whereas expression of dominant negative RASN17 was correlated with decreased sumoylation. Although sumoylation is not required for RAS activation it is necessary for its sustained activation and downstream signaling. Sumoylation was essential for activating RAS and its downstream signaling. Significantly, SUMO-resistant mutant of KRAS greatly reduced cellular migration and invasiveness in vitro and significantly suppressed tumor formation in vivo.

Extensive research in the past decades has shown that RAS proteins are extensively regulated at the post-translational levels (Ref. 1). However, to date SUMO modification has not been demonstrated for RAS although UBC9, the SUMO E2 conjugating enzyme in mammalian cells (Anckar and Sistonen, 2007), is required for oncogenesis driven by the RAS/Raf pathway (Yu et al.). Sumoylation plays a major role in regulating stability, activity, and subcellular localization and that UBC9, the SUMO E2 conjugating enzyme in mammalian cells UBC9, the SUMO E2 conjugating enzyme in mammalian cells (Anckar and Sistonen, 2007) is required for oncogenesis driven by the RAS/Raf pathway (Yu et al.).

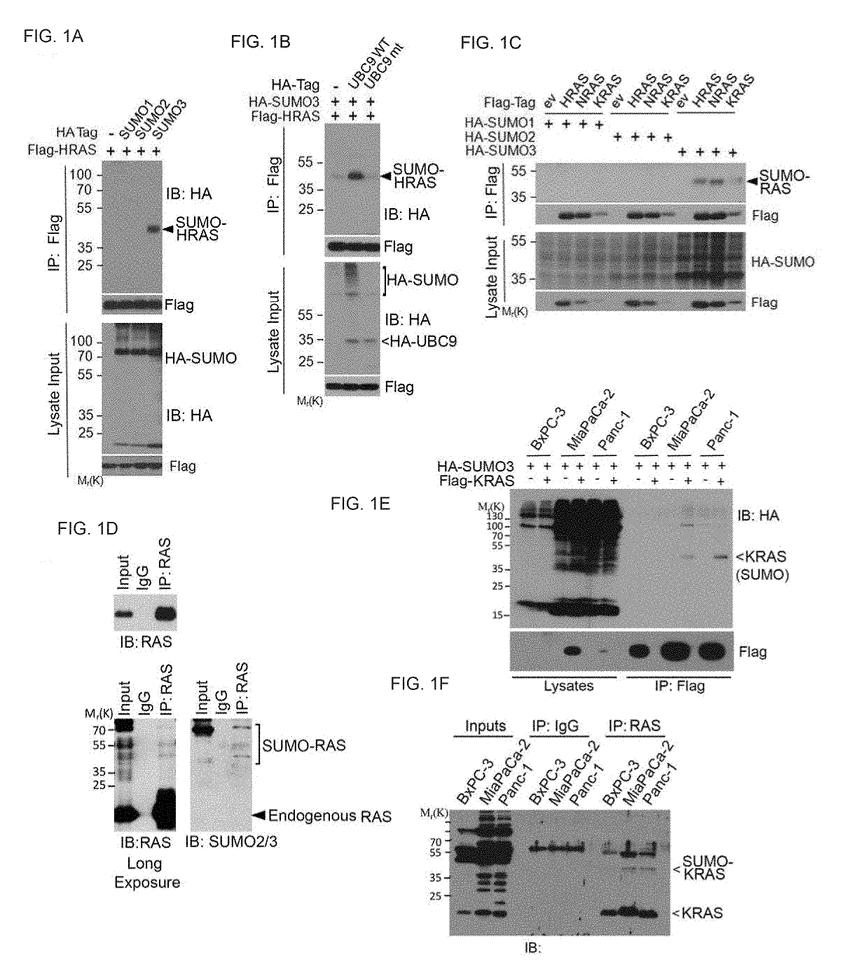

We investigated whether RAS proteins were post-translationally modified by SUMOs. Flag-HRAS and HA-tagged SUMO1, SUMO2, or SUMO3 were ectopically expressed. Flag immunoprecipitates contained significant amount of SUMO-HRAS in cells transfected with HA-SUMO3 and that the molecular weight was about the size of HRAS conjugated with one SUMO3 moiety (FIG. 1A). Expression of SUMO and UBC9 was confirmed by blotting with the anti-HA antibody. To confirm that HRAS was modified by SUMO3, cells with Flag-HRAS and HA-SUMO3 were co-transfected along with the wild-type (WT) or catalytically inactive mutant of UBC9. Flag immunoprecipitates contained significant amounts of SUMO3-modified HRAS when cells were transfected with WT UBC9, but not mutant UBC9 (FIG. 1B). HA-UBC9 expression was confirmed by blotting with the anti-HA antibody.

To determine whether SUMO-modification was unique to H-RAS, cells were transfected with Flag-KRAS4B (named KRAS thereafter), Flag-NRAS or Flag-HRAS expression constructs, along with HA-tagged SUMO1, SUMO2 or SUMO3. Immunoprecipitates of all three isoforms of RAS contained SUMO3, but not SUMO1- or SUMO2 (FIG. 1C), demonstrating that RAS proteins are primarily modified by SUMO3 in an isoform independent or dependent manner. Although the level of sumoylated KRAS was relatively low, this was consistent with the lower level of overall FLAG-KRAS expression. To determine whether endogenous RAS was also sumoylated, HEK293T cell lysates were immunoprecipitated with an anti-RAS antibody or control IgG. Blotting immunoprecipitates with both anti-RAS and anti-SUMO2/3 antibodies revealed that slow mobility RAS-specific bands were detectable that co-migrated with SUMO signals, strongly suggesting that these bands are SUMO-modified RAS (FIG. 1D).

KRAS plays a crucial role in mediating oncogenesis in pancreas (di Magliano and Logsdon). We next determined whether KRAS sumoylation occurred in pancreatic cell lines. BxPC-3, MiaPaCa-2, and Panc-1 cells were transfected with plasmids expressing Flag-KRAS and HA-SUMO3 for 24 h. Flag-immunoprecipitates were blotted with HA. We observed that sumoylation of ectopically expressed KRAS was easily detectable in MiaPaCa-2 and Panc-1 cells (FIG. 1E). Subsequent studies revealed that endogenous KRAS was sumoylated in all three pancreatic cell lines although SUMO-modified KRAS signals were weaker in BxPC-3 cells than those in MiaPaCa-2 and Panc-1 (FIG. 1F). Of note, BxPC-3, but not MiaPaCa-2 and Panc-1, cells contain WT KRAS (Fleming et al., 2005; Shen et al., 2012).

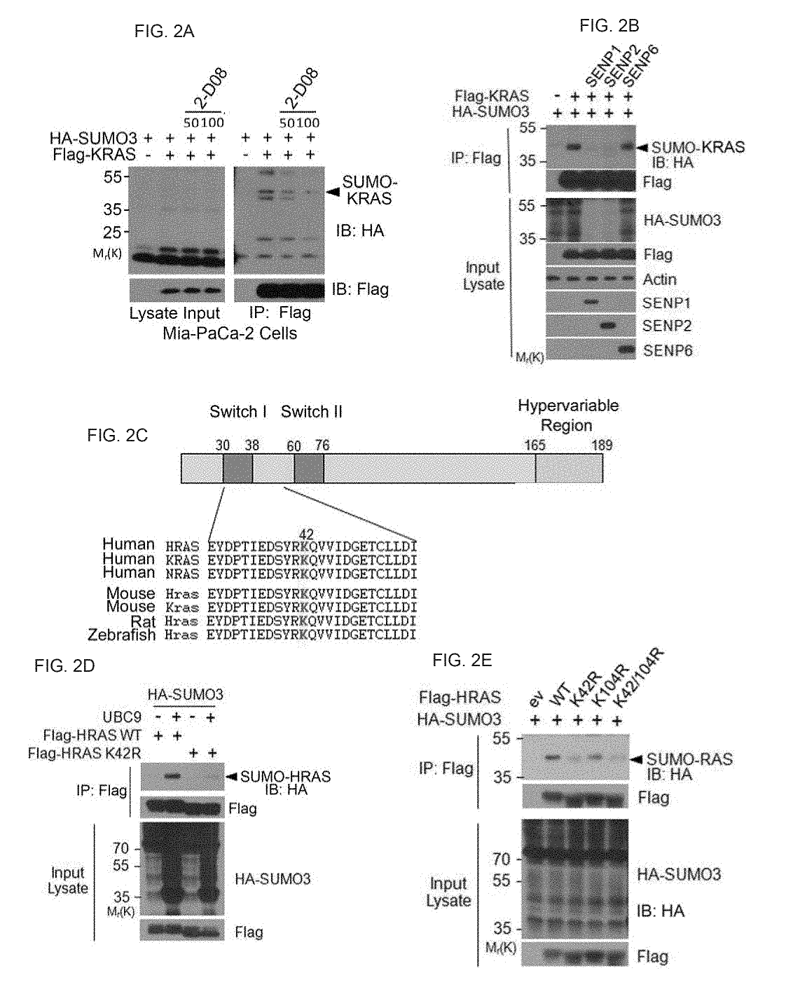

To further validate that KRAS was sumoylated, treated cells were transfected with Flag-KRAS expression plasmid with 2-D08, a SUMO E2 inhibitor (Kim et al., 2014). 2-D08 blocked KRAS sumoylation in a concentration-dependent manner (FIG. 2A). To determine whether KRAS sumoylation was a reversible process, cells were co-transfected with plasmid constructs expressing Flag-KRAS, HA-SUMO3, and an isopeptidase (SENP1, SENP2, or SENP6) that was capable of removing a specific SUMO moiety from its substrates (Kaikkonen et al., 2009). Expression of SENP1 or SENP2, but not SENP6, abolished sumoylated KRAS (FIG. 2B), indicating that SENP1 and SENP2 are likely isopeptidases that desumoylate KRAS in vivo.

To identify a potential lysine residue(s) that was SUMO-modified, we scanned RAS protein sequences optimal for sumoylation using online software (SUMOplot). We noted that K42 was one of two sites potential for sumoylation and that this residue was highly conserved (FIG. 2C). To test the hypothesis that K42 is the site of sumoylation of RAS we substituted arginine (R) for lysine (K) at this position and found a marked decrease/reduction in incorporation of HA-SUMO3 (FIG. 2D), confirming that K42 is the primary acceptor site. Because K104 is another site potential for sumoylation and this site is also known for RAS acetylation (Yang et al.), we asked whether K42 sumoylation was affected by the status of K104. K104 mutation alone somewhat compromised H-RAS sumoylation; however, additional K42 mutation did not further reduce residual signals of sumoylation (FIG. 2E). Expression of various mutants was comparable as revealed by blotting with the anti-Flag antibody.

### 6.2 Sumoylation is Associated with Ras Activation

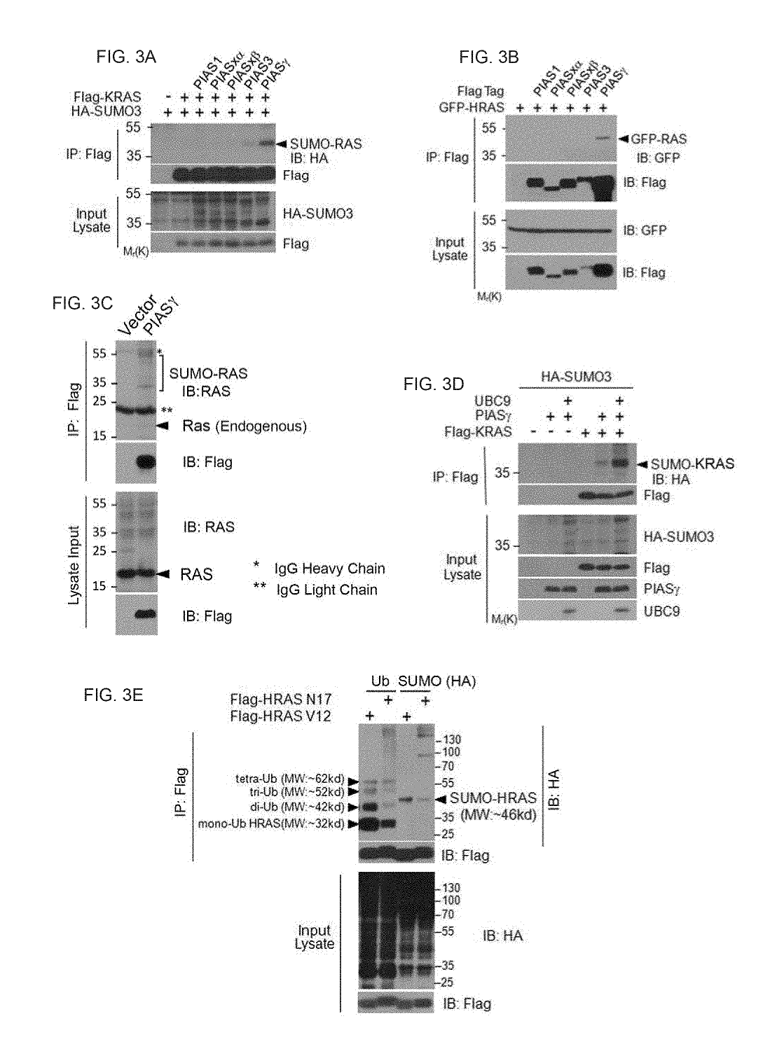

As RAS proteins can be modified by mono- and di-ubiquitination (Jura et al., 2006), we further investigated whether SUMO-modified RAS overlapped with any ubiquitinated forms. We co-transfected cells with Flag-HRASV12 or Flag-HRASN17 (a dominant negative mutant) and HASUMO3 or HA-ubiquitin. We observed that expression of HRASV12 resulted in higher levels of sumoylation than that of HRASN17 (FIG. 3E). Moreover, expression of HRASV12 resulted in more mono- and di-ubiquitinated forms of RAS proteins than those in cells expressing HRASN17. Additional modifications with high molecular weights were present in HA immunoprecipitates of cells expressing Flag-HRASN17. Significantly, SUMO-modified HRAS did not co-migrated with any of the ubiquitinated forms. Expression of these two mutant proteins was comparable as revealed by blotting with the anti-Flag antibody. Described herein is that SUMO-modified HRAS migrated at the position of about 46 kDa, we speculate that RAS can be di-sumoylated or mono-sumoylated but coupled with a ubiquitin moiety.

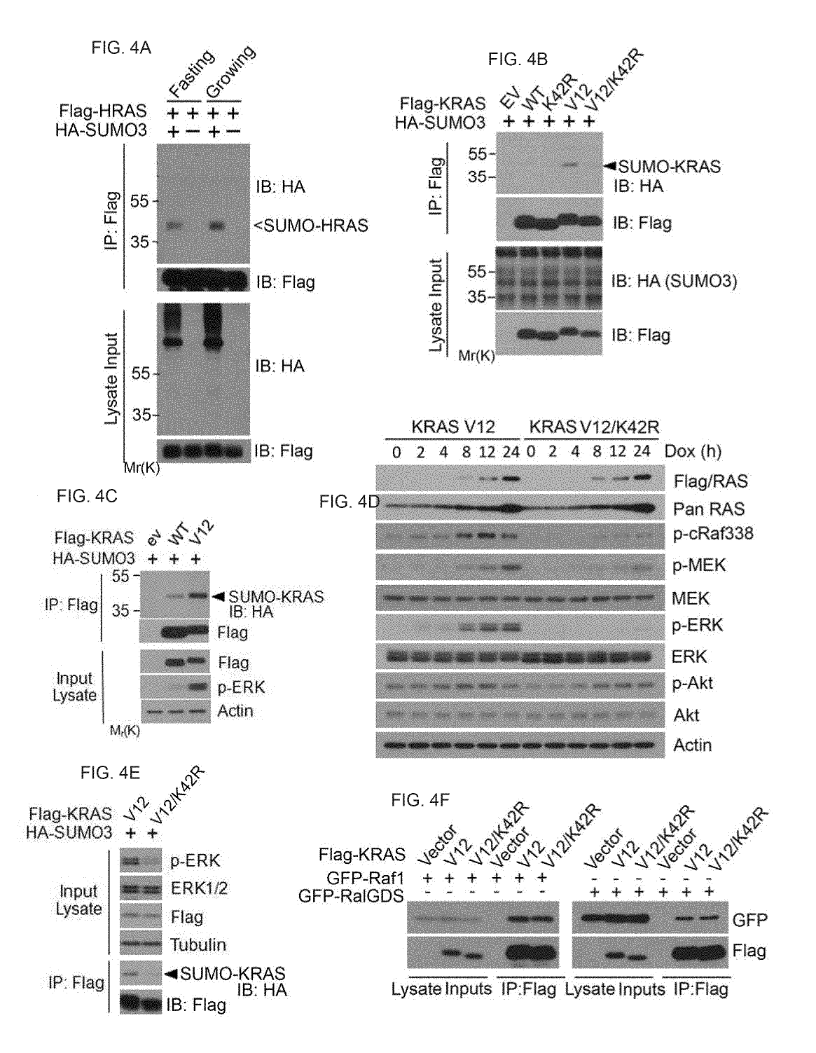

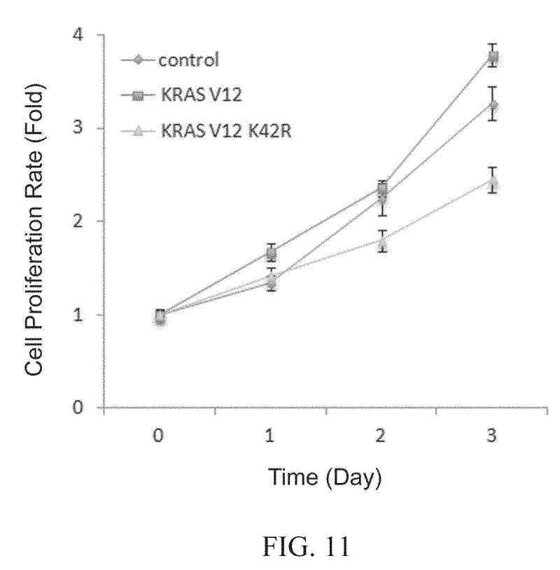

To study whether RAS sumoylation is associated with cell growth, we transfected cells with either Flag-H-RAS or its sumoylation-resistant mutant construct and analyzed the sumoylation status in both serum-starved and growing conditions. We observed that HRAS sumoylation was higher in rapid proliferating cells than in serum-starved cells (FIG. 4A). As KRASV12 played a key role in malignant transformation and tumorigenesis, we analyzed KRAS sumoylation in cells transfected with individual constructs expressing Flag-KRAS, Flag-KRASV12, or sumoylation-resistant mutant (i.e., Flag-KRASK42R or Flag-KRASV12/42R). Expression of mutant KRASV12 resulted in higher levels of sumoylation than that of WT KRAS, and mutation at K42 greatly reduced sumoylation of KRASV12 (FIG. 4B). Expression of various RAS proteins was comparable as revealed by blotting with the anti-Flag antibody. As expected, KRASV12 expression resulted in a higher level of ERK phosphorylation (p-ERK) than that of WT KRAS, even though its total protein level was lower (FIG. 4C). Significantly, compared with KRASV12, KRASV12/42R expression greatly reduced p-ERK signals despite that their expression was similar (FIG. 4E). Consistent with these observations, expression of KRASV12/42R substantially suppressed the proliferation rate as compared with that of Flag-KRAS42R (FIG. 11). As expected, mutation at K42 largely abolished K-RASV12 sumoylation. Combined, these studies suggest that K-RAS sumoylation plays an essential role in its activation, as well as the activation of cell proliferation pathway mediated by ERKs.

To further confirm that sumoylation at K42 promoted RAS activation and its downstream signaling, KRASV12 and KRASV12/42R expression was subjected to induction by doxycycline (Dox). We observed that expression of KRASV12 was strongly associated with the activation of downstream components including cRAF, MEK and ERK and that the activation of down-stream signaling components was KRAS concentration-dependent (FIG. 4E). On the other hand, KRASV12/42R greatly suppressed the activation of cRaf, MEK and ERK albeit its expression was comparable to KRASV12 after Dox induction, strongly supporting the notion that sumoylation is crucial for the activation of RAS, as well as its major signaling cascade of Raf/MEK/ERK. Since RAS is known to interact with PI3 kinase (Rodriguez-Viciana et al., 1997), we measured the activation kinetics of AKT after induced expression of KRASV12 and KRASV12/42R. Intriguingly, AKT activation was only slightly affected in cells expressing sumoylation-resistant mutant KRASV12/42R as compared with those expressing oncogenic KRASV12.

RAS protein residues between 32-40 (effector domain) are important in steering engagement with downstream effectors, leading to differential biological responses (Rodriguez-Viciana et al., 1997). As K42 is in a close proximity to the so-called “effector domain”, we determined whether K42 mutation would affect the interaction of RAS with down-stream effectors. Cells were co-transfected with Flag-KRASV12 or Flag-KRASV12/42R and GFP-Rafl (or GFPRal/GDS). Transfected cells were lysed and immunoprecipitated with the Flag antibody. We observed that Flag immunoprecipitates from cells expressing KRASV12 or KRASV12/42R contained roughly equal amounts of Rafl and Ral/GDS (FIG. 4F). These observations strongly suggest that K42 mutation did not significantly perturb conformation of RAS protein that would otherwise compromise physical interaction with its effectors.

To further confirm that sumoylation positively regulates RAS, we co-transfected cells with Flag-HRASV12 or Flag-HRASN17 (a dominant negative mutant) and HA-SUMO3 or HA-ubiquitin. We observed that expression of H-RASV12 resulted in higher levels of sumoylation than that of H-RASN17 (FIG. 3E). Moreover, expression of H-RASV12 resulted in more mono- and di-ubiquitinated forms of RAS proteins than those in cells expressing H-RASN17. Intriguingly, additional modifications with high molecular weights were present in HA immunoprecipitates of cells expressing Flag-H-RASN17. Expression of these two mutant proteins was comparable as revealed by blotting with the anti-Flag antibody.

### 6.3 PIASγ is a SUMO E3 Ligase for RAS Proteins

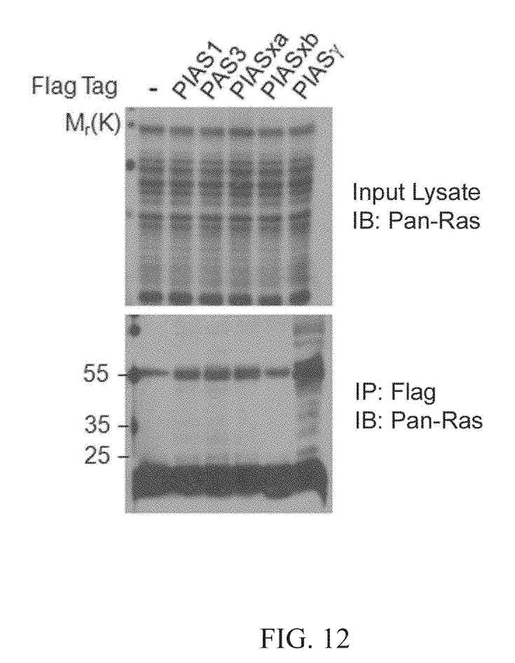

To identify a potential SUMO E3 ligase(s) for RAS, we ectopically expressed various genes of the PIAS family (Palvimo, 2007; Rytinki et al., 2009) and determined which gene product(s) was capable of stimulating K-RAS sumoylation. We observed that expression of PIASγ significantly stimulated K-RAS sumoylation although PIAS3 also induced a low level of sumoylation (FIG. 3A), suggesting that PIASγ may be a likely SUMO E3 for K-RAS. Consistent with this observation, PIASγ (PIAS4) is required for conjugating SUMO2/3 to protein substrates during DNA damage responses (Galanty et al., 2009). Expression of KRAS and various PIAS family members was comparable as revealed by blotting with the anti-Flag antibody. PIASγprecipitates, but not pull-down materials of other members of the PIAS family, contained significant amounts of HRAS signals (FIG. 3B), suggesting the physical interaction between HRAS and PIASγ. Specific interaction between K-RAS and PIASγ was also detected (FIG. 3B and FIG. 12). Moreover, ectopically expressed PIASγ was capable of immunoprecipitating endogenous RAS protein (FIG. 3C). We then investigated whether PIASγ was capable of specific stimulating KRAS sumoylation. Expression of PIASγ enhanced KRAS sumoylation, which was further stimulated by expression of UBC9 (FIG. 3D), strongly suggesting that PIASγis a bona fide SUMO E3 ligase for RAS proteins. Further studies revealed that SENP1 and SENP2 isopeptidases, but not SENP6 isopeptidase, were capable of removing the SUMO moiety from K-RAS (FIG. 2B).

To further study whether sumoylation was a critical step in the maintenance of RAS activity and its signaling, HeLa cells were co-transfected for 24 h with expression plasmids for CFP-H-RAS and GFP-Rafl RBD (RAS binding domain), along with UBC9 expression plasmid or control plasmid. Transfected cells were then serum-starved overnight. Before the addition of fetal bovine serum, CFP-H-RAS in cells with or without ectopically expressed UBC9 was largely distributed throughout the cell. Serum treatment significantly induced the association of CFP-H-RAS to the cell surface membrane, which was further enhanced by expression of UBC9, suggesting that sumoylation plays a role in the activation of H-RAS. We then transfected HeLa cells with a plasmid construct expressing either H-RAS or its sumoylation-resistant mutant, along with CFP-HRAS and GFP-Rafl RBD. We observed that expression of WT H-RAS significantly enhanced CFP-HRAS association with the plasma membrane whereas expression of H-RASK42R did not have the same effect. This observation further supports the notion that RAS sumoylation is important for its activation through the association with the cell surface membrane.

### 6.4 SUMO-Resistant KRAS Mutant Suppresses Cell Migration and Invasion

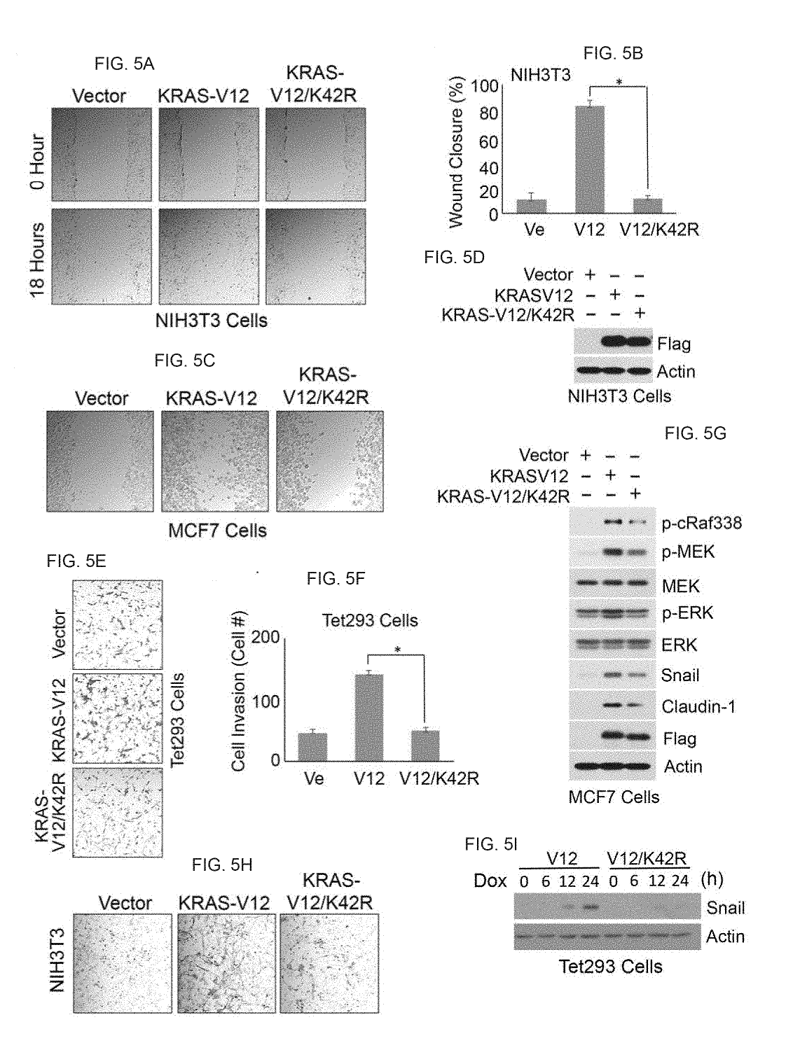

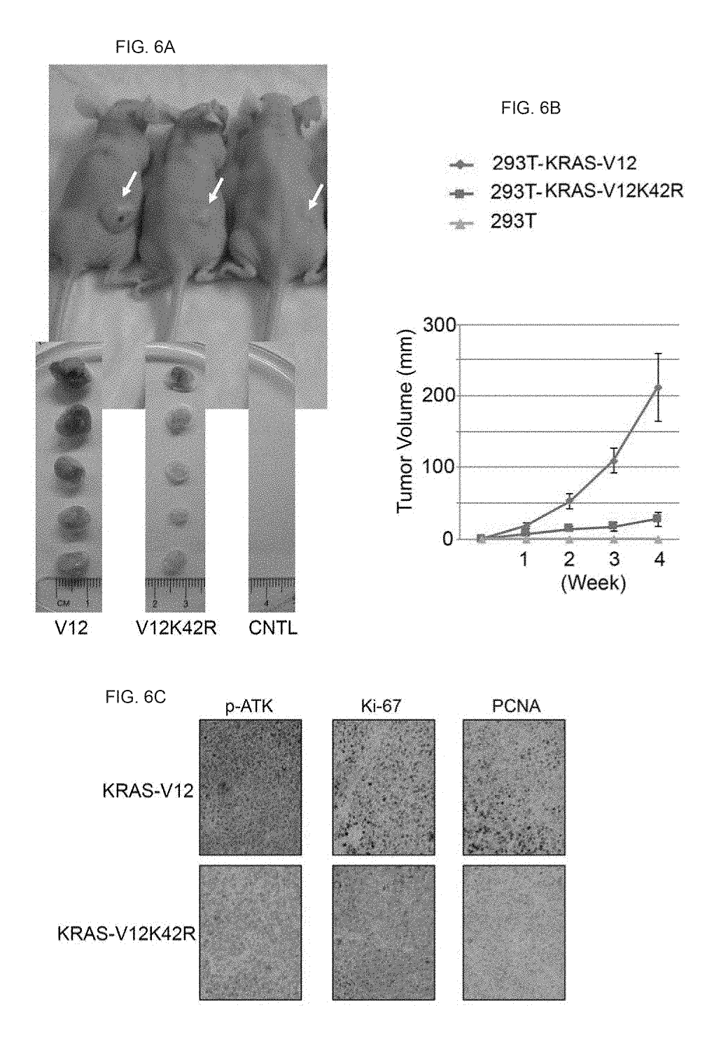

KRAS oncogenic mutations (e.g., V12) occur early in carcinogenesis of major human malignancies, which is known to promote cell migration and invasion of cancer cells (Tsai et al.; Yu et al.). We then measured whether K42R mutation would affect cell migration promoted by KRASV12 in a conventional wound-healing assay. We observed that NIH3T3 cells transfected with Flag-KRASV12 displayed rapid closing of wound gap due to active cell migration compared with that of cells transfected with Flag-KRASV12/42R or vector alone (FIGS. 5A and 5B), strongly suggesting that sumoylation-resistant KRASV12 significantly compromises its ability to promote cell migration. Expression of transfected RAS was efficient (FIG. 5C). To confirm that sumoylation plays a role in cell migration, we ectopically expressed KRASV12 or KRASV12/42R in MCF7 cells. We observed that expression of sumoylation-resistant mutant also suppressed MCF7 cell migration promoted by KRASV12 (FIG. 5D). This suppression was associated with down-regulation of the MAK kinase signaling pathway coupled with reduced expression of Snail and Claudin-1 (FIG. 5E), two gene products involved in cell migration and epithelial and mesenchymal transition (EMT) (Shih and Yang, 2011). Expression of SUMO-resistant KRAS mutant also somewhat affected cell proliferation, which did not seem to be associated with increased cell death or cell cycle arrest (FIG. 6 and FIG. 11).

We further examined whether KRAS sumoylation affected its ability to promote cell invasion. Tet293 cells stably transfected with KRASV12 or KRASV12/42R expression plasmid, along with parental cells, were subjected to transwell migration assay. We observed that compared with the parental control cells, significantly increased invasiveness was observed in Tet293-KRASV12 cells and that Tet293-KRASV12/42R cells exhibited greatly reduced transwell migration similar to that of parental cells (FIG. 5F and FIG. 5G). Consistent with these observation, transfection of KRASV12 expression plasmid, but not KRASV12/42R or vector, greatly promoted transwell migration of NIH3T3 cells (FIG. 5H). Moreover, addition of Dox to Tet293-KRASV12 cells, but not Tet293-KRASV12/42R cells, induced expression of Snail in a time-dependent manner (FIG. 5I). Combined, these observations strongly suggest that sumoylation plays an essential part in oncogenic activities of KRASV12.

### 6.5 SUMO-Resistant KRAS Mutant Compromises Tumor Development in Nude Mice

To further determine whether sumoylation played an essential role in tumor development caused by oncogenic activation of KRAS, we carried out mouse xenograft experiments by inoculating nude mice with cells expressing either KRASV12 or its SUMO-resistant mutant (KRASV12/42R). We observed that nude mice subcutaneously injected with cells expressing both KRASV12 and KRASV12/42R developed tumors (FIG. 6A). However, tumors formed from KRASV12-expressing cells were significantly larger than those from cells expressing KRASV12/42R (FIGS. 6A and 6B), strongly suggesting that sumoylation-resistant KRAS mutant displays a weakened oncogenic activity compared with KRASV12 counterpart. Consistent with this notion, cell proliferation markers including p-AKT, Ki-67, and PCNA were expressed at much higher levels in KRASV12-expressing tumors than those of KRASV12/42R-expressing tumors (FIG. 6C).

### 6.6 SUMO Inhibitor Blocks Cell Migration in KRAS-Dependent Manner

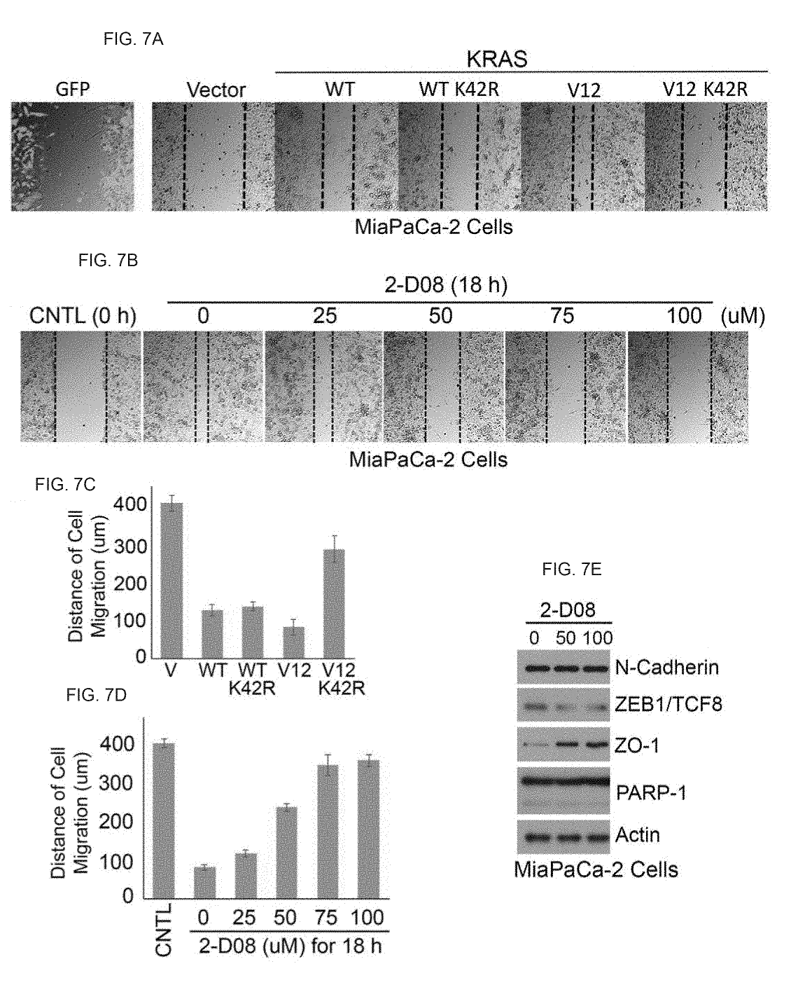

RAS proteins were the first, and remain the best-studied, oncoproteins. However, to date no compounds that target RAS have been approved for clinic applications for cancer treatment. As a proof-of-principle study, we next determined whether 2-D08 that blocks RAS sumoylation (FIG. 2A) would suppress tumor cell migration in a KRAS-dependent manner. Wound-healing assays revealed that oncogenic KRAS substantially promoted pancreatic cancer cell migration whereas the sumoylation-resistant mutant suppressed cell migration in both WT KRAS and oncogenic KRAS (KRASV12) (FIGS. 7A & 7C). Gene transfection was efficient as revealed by high levels of expression of co-transfected GFP in these cells. Treatment with 2-D08, a SUMO E2 inhibitor (Ref 35), blocked MiaPaCa-2 cell migration in a concentration-dependent manner (FIG. 7B & 7D). However, 2-D08 did not have an effect on inhibiting cell migration of BxPC-3 cells that harbor no KRAS mutation. Moreover, treatment of MiaPaCa-2 cells with 2-D08 suppressed expression of ZEB1/TCF8 (a gene product positively associated with cell migration and EMT transition) whereas it promoted expression of ZO-1 (a gene product whose expression is negatively associated with cell proliferation and migration) (FIG. 7E). These results suggest that sumoylation in general and KRAS sumoylation in particular can be explored as a potential new target for anticancer drug development.

### 6.7 RAS Sumoylation and Downstream Signaling

RAS proteins are among the most potent oncogenic gene products, which directly impact upon the MAP kinase signaling pathway. In this study, we report for the first time that RAS proteins can be modified by sumoylation and that sumoylation is important for the activation of RAS proteins and its downstream signaling, and oncogenic activities. We have observed that there are several salient features of RAS sumoylation. (1) All three isoforms of RAS proteins (HRAS, KRAS and NRAS) are sumoylated; (2) SUMO3, but not SUMO1 or SUMO2, is conjugated to RAS proteins; (3) sumoylation occurs on a single lysine residue (K42) as mutation of this residue into arginine largely abolishes its sumoylation; (4) RAS sumoylation is a general phenomenon, occurring in all cell types that are examined; (5) Moreover, RAS sumoylation greatly impacts on its activity. Expression of sumoylation-resistant KRAS significantly suppresses cell migration and invasion hi vitro and tumorigenesis hi vivo.

RAS sumoylation on K42 is directly linked to its activation as sumoylation-resistant mutant suppresses its downstream signaling, leading to greatly reduced activities of cRaf, MEK and ERK. This is significant considering that RAS proteins have been subjected to extensive studies in the past and that RAF/MEK/ERK comprises the classical pathway for regulating cell proliferation, differentiation, and oncogenesis. At present, we cannot exclude a remote possibility that K42 mutation itself would somehow change downstream signaling due to major structural changes. However, available evidence strongly suggests that substitution of K42 with R42 does not perturb its conformation. (1) Physical interaction between KRASV12/42R and Rafl or Ral.GDS is not compromised when it is compared to that of KRASV12 (FIG. 4F); (2) KRASV12/42R regulates AKT signaling/activation in a manner similar to that of KRASV12 (FIG. 4E). Consistent with our findings, it has been shown that RASV12/42A interacts with Ral.GDS, Raf , and PI3-kinase in the same manner as that of wild-type RAS or RASV12 mutant regardless of GTP loading (Rodriguez-Viciana et al., 1997). Of note, sumoylation occurs frequently at non-consensus motifs of many proteins (Anckar and Sistonen, 2007). It is generally agreed that SUMO E2 (UBC9) alone is capable of promoting sumoylation and that SUMO E3 ligases function to enhance the efficiency and substrate-specificity of sumoylation, especially for those non-consensus sites. Supporting the notion, we have observed that although PIASγ alone enhances KRAS sumoylation, inclusion of UBC9 significantly promotes the process (FIG. 3D). The stoichiometry of constitutive SUMO-modification of RAS proteins is not high (FIGS. 1D and 1F) and UBC9 and/or PIASγ can significantly boost RAS sumoylation (FIGS. 3C and 3D). We propose that only a small pool of total RAS (e.g., KRAS) proteins are biologically active and that this pool is sumoylated. Sumoylation positively regulate the association of RAS with the plasma membrane. Specifically, we have shown that UBC9 promotes the association of RAS with the plasma membrane after serum stimulation, strongly suggesting sumoylation in RAS activation. Significantly, we have demonstrated that different from WT RAS, sumoylation-resistant RAS is not enriched at the plasma membrane.

Extensive research in the past has revealed that RAS is distributed within the cell through the extensive membrane trafficking process (Ref. 1, 25). Post-translational processes play a pivotal role during the process. Farnesylated RAS proteins gain modest affinity with membranes. It is believed that additional modifications including palmitoylation and acylation are essential steps for translocation to the plasma membrane (Ref. 1, 25). At present, it is unclear whether RAS sumoylation occurs in endoplasmic reticulum and/or in the Golgi complex. Described herein is that PIASγ is a sumoylation E3 ligase of RAS, it would be interesting to determine whether this enzyme is enriched or localized in a particular cellular organelle(s). It has been shown that N-RAS and H-RAS, but not K-RAS, traffic from the Golgi to the plasma membrane via the recycling endosomes (Ref. 1). We have observed that all three forms of RAS proteins are sumoylated, suggesting that sumoylation may occur either at an early stage after protein synthesis or at the plasma membrane.

We have observed that compared with that of dominant negative form of HRASN17, activated H-RASV12 exhibits enhanced sumoylation, as well as elevated levels of mono- and di-ubiquitination. Since HRAS is activated by monoubiquitination and ubiquitination accelerates intrinsic nucleotide exchange, thereby promoting GTP loading (Jura et al., 2006; Sasaki et al.), our observation suggests that GTP loading can play a role in modulating its sumoylation. Alternatively, sumoylation may facilitate the plasma membrane and/or endosome association of RAS, leading to its activation. It has been shown that among the isoforms of RAS, there are differences in the mode of actions in their activation by monoubiquitination (Pfleger; Sasaki et al.). HRAS activity is promoted by monoubiquitination at K117 through accelerating intrinsic nucleotide exchange, leading to enhanced GTP loading; on the other hand, KRAS monoubiquitination at K147 results in impaired GTP hydrolysis (Pfleger; Sasaki et al.). The net effect, therefore, remains the same: the activation of RAS protein. K42 is a highly conserved a residue among all three RAS isoforms. Since HRAS, KRAS and NRAS can be all modified by SUMO, we speculate that sumoylation may represent a fundamental mechanism by which RAS activities and/or their subcelluar localization is controlled. Similar to di-ubiquitination, modification with SUMO3 is likely to affect subcellular localization of RAS proteins.

KRAS is the major driver of human cancers. It is generally agreed that KRAS G12V mutant is constitutively locked in the active, GTP-bound state, which makes it very challenging to inhibit its oncogenic activity through traditional drug design approaches. It appears feasible to disrupt the association between RAS and its downstream effectors via a small molecular compound, thus blocking its signaling and activity (Athuluri-Divakar et al., 2016). In the current study, we demonstrate that modulation of K42 through suppressing sumoylation can significantly impact on the activity of KRASV12, especially with regard to its ability to drive cell migration and invasion in vitro and tumorigenesis in vivo (FIGS. 5A-5I and FIGS. 6A-6C). This is extremely significant as regulatory components of sumoylation provide new opportunities for drug design and cancer intervention. For example, PIASγ, a SUMO E3 ligase for RAS proteins, is druggable and can be explored as a new target for discovery and development of small molecular compounds for major human malignancies of lung and pancreas. Supporting this, we have observed that PIASγ is overexpressed in human pancreatic cancer specimens. Moreover, we have demonstrated that SUMO inhibitor 2-D08 suppresses expression of genes involved in EMT and reduces migration rate of pancreatic cancer cells with KRASV12 in a concentration-dependent manner (FIG. 7). Described herein is that sumoylation closely resembles ubiquitination and that small molecular inhibitors for ubiquitin and the proteasome have been moved to the clinic for cancer treatments (Landre et al., 2014; Niewerth et al., 2013), we believe that a better understanding of the role of sumoylation in regulating RAS proteins will open a new path to develop innovative anti-cancer therapies.

### 6.8 Amino Acid Mutations and Substitutions in RAS

Methods for engineering a mutation or substitution into the primary amino acid sequence of a RAS protein are well known in the art via standard techniques. The RAS proteins for use in the methods described herein may include conservative substitutions. A “conservative amino acid substitution” is one in which the amino acid residue is replaced with an amino acid residue having a side chain with a similar charge. A “non-conservative amino acid substitution” is one in which the amino acid residue is replaced with an amino acid residue having a side chain with an opposite charge. Families of amino acid residues having side chains with similar charges have been defined in the art. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine (Ref. 43).

Substantial modifications in the biological properties of RAS proteins are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Non-conservative substitutions will entail exchanging a member of one of these classes for another class.

### 6.9 Materials and Methods

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, for example, “Molecular Cloning: A Laboratory Manual”, second edition (Ref. 42).

### 6.9.1 Cell Culture and Transfection

HeLa (cervical carcinoma), HEIS-293T (kidney carcinoma), MCF7 (mammary carcinoma), and NIH3T3 (fibroblast) cell lines obtained from the American Type Culture Collection were cultured in DMEM supplemented with 10% fetal bovine serum (FBS, Invitrogen) and antibiotics (100 μg/ml of penicillin and 50 μg/ml of streptomycin sulfate, Invitrogen) at 37° C. under 5% CO2. Transfection of HeLa cells or HEK293T was achieved with either LF2000 (Invitrogen) or Fugene HD (Roche Diagnostics) following the manufacturers' protocol. Transfection efficiency was estimated to be between 80-100% in all cases through transfecting a GFP expressing plasmid (Data not shown).

### 6.9.2 Plasmids

HA-tagged SUMO1, SUMO2, SUMO3, wild-type UBC9, UBC9 mutant, Flag-tagged PIAS1, PIASxα, PIASxβ, PIASγ, SENP1, SENP2 and SENP6 were obtained from Addgene. Flag-PIAS3 was kind gifts from Dr. Angeliki Malliri. Individual RAS mutants were obtained using Quikchange Site-directed mutagenesis kit (Agilent Technologies). All mutations were confirmed by DNA sequencing.

### 6.9.3 Protein Extracts and Immunoblotting

Total cell lysates were prepared in a buffer [50 mM Tris-HCl (pH 7.5), 150 mM NaCl, 1% IGEPAL, 0.1% SDS, and 0.5% sodium deoxycholate] supplemented with a mixture of protease and phosphatase inhibitors. HEK293T Tet-on cells were engineered to express KRASV12 or KRASV12/42R mutants in the presence of doxycycline. At various times post addition of doxycycline, cells were collected and lysed in the same buffer as above. Protein concentrations were measured using the bicinchoninic acid protein assay reagent kit (Pierce Chemical Co). Equal amounts of protein lysates from various samples were used for SDS-PAGE analysis followed by immunoblotting. Antibodies to HA, Flag, UBC9, PIASγ, PIASγphosphocRaf, Rafl, Ral.GDS, phospho-MEK, MEK, phospho-ERK 42/44, ERK1/2, RAS phospho-AKT, AKT, and Actin were purchased from Cell Signaling Technology. Antibody to SENP1, SENP2 and SENP6 were purchased from Santa Cruz Biotechnology. Specific signals on immunoblots (polyvinylidene difluoride) were visualized using enhanced chemiluminescence (Super-Signal, Pierce Chemical Co.).

### 6.9.4 Immunoprecipitation

HEK-293T cells were lysed in TBSN buffer [20 mM Tris-Cl (pH 8.0), 150 mM NaCl, 0.5% NP-40, 5 mM EGTA, 1.5 mM EDTA, 0.5 mM Na3VO4, and 201 M □-Glycerol phosphate]. The cell lysates were clarified by centrifugation at 15,000×g for 20 min at 4° C. Cleared lysates (1 mg) were added to Flag M2 agarose (Sigma) followed by incubation in the TBSN buffer for 1 h at 4° C. After incubation, proteins bound to each resin were washed extensively with the binding buffer, eluted in the SDS-PAGE sample buffer, and analyzed by SDSPAGE.

### 6.9.5 Imaging and Stimulation

HeLa cells were seeded on chamber slides and co-transfected with CFP-H-RAS expression construct (or mutants) and GFP-Rafl RBD for 24 h. Cells were then starved with 0.5% serum containing medium for 16 h. To maintain appropriate pH and avoid evaporation, CO2-independent medium (Invitrogen) supplemented with 10% FBS and antibiotics was used. Cells were fixed with 4% paraformaldehyde. After three washes with 0.1% Triton X-100 in PBS, DNA was stained with DAPI. Confocal microscopy (Leica TCS SP5) was used to record fluorescent images.

### 6.9.6 Wound-Healing Assay

NIH3T3 fibroblast cells were transiently transfected with plasmid vector alone or pcDNA-KRASV12 or pcDNA-KRASV12/42R for 24 h before they were used for wound-healing assay. Transfected cells were serum-starved for 18 h and then wounded by manual scraping with a pipette tip. Plates were washed several times to remove non-adherent cells and cells were cultured in the same medium containing 1% FBS. Assessment of cell migration into the wounded area was performed under a light microscope after additional 18 h of incubation.

### 6.9.7 Cell Invasion Assays

MDA231, HEK293T, and NIH3T3 cells were transiently transfected with pcDNA plasmid vector (control), pcDNA-KRASV12 or pcDNA-KRASV12/42R. Twenty-four h post transfection, cells (4×104 cells/well) were seeded onto transwell inserts and incubated at 37° C. for 12 h. Non-migrated cells were removed from the upper face of the transwell insert using a cotton swab. Cells that migrated through membranes were stained. Cell density in each treatment was recorded under a light microscope.

### 6.9.8 Nude Mouse Xenograft Study

Six-week old female athymic nude mice were obtained from the Jackson Laboratory. The mice were maintained in a pathogen-free environment and all the procedures were carried out as per the protocol approved by the Institutional Animal Care and Use Committee (IACUC). Mouse xenograft models were made by subcutaneous heterotransplantation of parental HEK293T cells or HEK293T cells expressing KRASV12 or KRASV12K42R. Briefly, HEK293 cells or their derivatives (1×106) were dispersed in 100 μl PBS (1×) and 100 μl Matrigel (BD Biosciences) and inoculated subcutaneously into the right lower back region of BalB/c nude mice. Doxycycline were injected intraperitoneally every three days for 4 weeks. Once the tumor xenografts emerged, their sizes were measured once every other day until four weeks when the mice were sacrificed. Tumors formed in mice were excised, fixed, and embedded in paraffin. Embedded tumor samples were sectioned (5 micron) for immunohistochemistry analysis.

### 6.9.9 Statistical Analysis

Each experiment was performed at least three times. The data were plotted as the mean±S.D. Student's t-test was used for all comparisons. A P value of less than 0.05 was considered statistically significant.

### 7. EXAMPLE 2

A Ras protein (e.g., a sumoylated Ras protein), or a fragment thereof (e.g., an epitope comprising amino acid residue 42 of a Ras protein where amino acid residue 42 may be sumoylated or non-sumoylated) will be used to immunize mice intraperitoneally or intravenously. One or more boosts may or may not be given. The titers of the antibodies in the plasma can be monitored by, e.g., ELISA (enzyme-linked immunosorbent assay) or flow cytometry. Mice with sufficient titers of anti-Ras antibodies (or antibodies specific to a fragment of Ras, e.g., an epitope comprising amino acid residue 42 of a Ras protein where amino acid residue 42 may be sumoylated or non-sumoylated) will be used for fusions. Mice may or may not be boosted with antigen 3 days before sacrifice and removal of the spleen. The mouse splenocytes will be isolated and fused with PEG to a mouse myeloma cell line. The resulting hybridomas will then be screened for the production of antigen-specific antibodies. Cells will be plated, and then incubated in selective medium. Supernatants from individual wells will then be screened by ELISA for antigen-specific monoclonal antibodies (such as an antibody that specifically binds to a Ras protein, or a fragment thereof, e.g., an epitope comprising amino acid residue 42 of a Ras protein where amino acid residue 42 may be sumoylated or non-sumoylated). The antibody secreting hybridomas will be replated, screened again, and if still positive for anti-antigen monoclonal antibodies (such as an antibody that specifically binds to a Ras protein, or a fragment thereof, e.g., an epitope comprising amino acid residue 42 of a Ras protein where amino acid residue 42 may be sumoylated or non-sumoylated), can be subcloned by limiting dilution.

In one embodiment, the immunogen is a full-length Ras protein sumoylated at amino acid residue 42. The Ras protein may be a wild-type Ras protein or a mutant Ras protein as described herein. In one embodiment, the immunogen is a recombinant Ras fragment, such as fragments comprising (consisting of, or consisting essentially of) residues 1-166 where residue 42 is sumoylated or non-sumoylated. In certain embodiments, the immunogen is a fragment of a Ras protein having about 180 amino acid residues, about 160 amino acid residues, about 140 amino acid residues, about 120 amino acid residues, about 100 amino acid residues, about 80 amino acid residues, about 60 amino acid residues, about 40 amino acid residues, about 20 amino acid residues, or about 10 amino acid residues, where the fragment comprises amino acid residue 42 of a Ras protein, and where amino acid residue 42 may be sumoylated or non-sumoylated.

The disclosure is not to be limited in scope by the specific embodiments described herein. Indeed, various modifications of the disclosure in addition to those described will become apparent to those skilled in the art from the foregoing description and accompanying figures. Such modifications are intended to fall within the scope of the appended claims.

All references cited herein are incorporated herein by reference in their entireties and for all purposes to the same extent as if each individual publication or patent or patent application was specifically and individually indicated to be incorporated by reference in its entirety for all purposes.

### REFERENCES

1. Ahearn I M, Haigis K, Bar-Sagi D, Philips M R. Regulating the regulator: post-translational modification of RAS. Nat Rev Mol Cell Biol; 13:39-51.
2. Shaw R J, Cantley L C. Ras, PI(3)K and mTOR signalling controls tumour cell growth. Nature 2006; 441:424-30.
3. Hall A. The cellular functions of small GTP-binding proteins. Science 1990; 249:635-40.
4. Boguski M S, McCormick F. Proteins regulating Ras and its relatives. Nature 1993; 366:643-54.
5. Yaswen P. Campisi J. Oncogene-induced senescence pathways weave an intricate tapestry. Cell 2007; 128:233-4.
6. Sternberg P W, Alberola-Ila J. Conspiracy theory: RAS and RAF do not act alone. Cell 1998; 95:447-50.
7. Lynch S J, Snitkin H, Gumper I, Philips M R, Sabatini D, Pellicer A. The differential palmitoylation states of N-Ras and H-Ras determine their distinct Golgi subcompartment localizations. J Cell Physiol; 230:610-9.
8. Sung P J, Tsai F D, Vais H, Court H, Yang J, Fehrenbacher N, Foskett J K, Philips M R. Phosphorylated K-Ras limits cell survival by blocking Bcl-xL sensitization of inositol trisphosphate receptors. Proc Natl Acad Sci U S A; 110:20593-8.
9. Philips M R, Cox A D. GeranylgeranyltransfeRase I as a target for anti-cancer drugs. J Clin Invest 2007; 117:1223-5.
10. Bivona T G, Quatela S E, Bodemann B O, Ahearn I M, Soskis M J, Mor A, Miura J, Wiener H H, Wright L, Saba S G, Yim D, Fein A, Perez de Castro I, Li C, Thompson C B, Cox A D, Philips M R. PKC regulates a famesyl-electrostatic switch on K-Ras that promotes its association with Bcl-XL on mitochondria and induces apoptosis. Mol Cell 2006; 21:481-93.
11. Bar-Sagi D, Hall A. Ras and Rho GTPases: a family reunion. Cell 2000; 103:227-38.
12. Jura N, Scotto-Lavino E, Sobczyk A, Bar-Sagi D. Differential modification of Ras proteins by ubiquitination. Mol Cell 2006; 21:679-87.
13. Xu L, Lubkov V. Taylor L J, Bar-Sagi D. Feedback regulation of Ras signaling by Rabex-5-mediated ubiquitination. Curr Biol; 20:1372-7.
14. Yang M H, Nickerson S, Kim E T, Liot C, Laurent G, Spang R, Philips M R, Shan Y, Shaw D E, Bar-Sagi D, Haigis M C, Haigis K M. Regulation of RAS oncogenicity by acetylation. Proc Natl Acad Sci U S A; 109:10843-8.
15. Henis Y I, Hancock J F, Prior I A. Ras acylation, compartmentalization and signaling nanoclusters (Review). Mol Membr Biol 2009; 26:80-92.
16. Lander H M, Hajjar D P, Hempstead B L, Mirza U A, Chait B T, Campbell S, Quilliam L A. A molecular redox switch on p21(Ras). Structural basis for the nitric oxide-p21(Ras) interaction. J Biol Chem 1997; 272:4323-6.
17. Raines K W, Cao G L, Lee E K, Rosen G M, Shapiro P. Neuronal nitric oxide synthase-induced S-nitrosylation of H-Ras inhibits calcium ionophore-mediated extracellular-signal-regulated kinase activity. Biochem J 2006; 397:329-36.
18. Palancade B, Doye V. Sumoylating and desumoylating enzymes at nuclear pores: underpinning their unexpected duties? Trends Cell Biol 2008; 18:174-83.
19. Di Bacco A, Gill G. SUMO-specific proteases and the cell cycle. An essential role for SENP5 in cell proliferation. Cell Cycle 2006; 5:2310-3.
20. Dawlaty M M, Malureanu L, Jeganathan K B, Kao E, Sustmann C, Tahk S, Shuai K, Grosschedl R, van Deursen J M. Resolution of sister centromeres requires RanBP2-mediated SUMOylation of topoisomeRase Ilalpha. Cell 2008; 133:103-15.
21. Anckar J, Sistonen L. SUMO: getting it on. Biochem Soc Trans 2007; 35:1409-13.
22. Yu B, Swatkoski S, Holly A, Lee L C, Giroux V, Lee C S, Hsu D, Smith J L, Yuen G, Yue J, Ann D K, Simpson R M, Creighton C J, Figg W D, Gucek M, Luo J. Oncogenesis driven by the Ras/Raf pathway requires the SUMO E2 ligase Ubc9. Proc Natl Acad Sci U S A; 112:E1724-33.
23. Rytinki M M, Kaikkonen S, Pehkonen P, Jaaskelainen T, Palvimo J J. PIAS proteins: pleiotropic interactors associated with SUMO. Cell Mol Life Sci 2009; 66:3029-41.
24. Palvimo J J. PIAS proteins as regulators of small ubiquitin-related modifier (SUMO) modifications and transcription. Biochem Soc Trans 2007; 35:1405-8.
25. Mor A, Philips M R. Compartmentalized Ras/MAPK signaling. Annu Rev Immunol 2006; 24:771-800.
26. Sasaki A T, Carracedo A, Locasale J W, Anastasiou D, Takeuchi K, Kahoud E R, Haviv S, Asara J M, Pandolfi P P, Cantley L C. Ubiquitination of K-Ras enhances activation and facilitates binding to select downstream effectors. Sci Signal; 4:ra13.
27. Pfleger C M. Ubiquitin on Ras: warden or partner in crime? Sci Signal; 4:pe12.
28. Lu P D, Jousse C, Marciniak S J, Zhang Y, Novoa I, Scheuner D, Kaufman R J, Ron D, Harding H P. Cytoprotection by pre-emptive conditional phosphorylation of translation initiation factor 2. EMBO J 2004; 23:169-79.
29. Xu D, Yao Y. Jiang X, Lu L, Dai W. Regulation of PTEN stability and activity by PLK3. J Biol Chem. 2010, 285(51):39935-42.
30. Athuluri-Divakar, S. K., Vasquez-Del Carpio, R Dutta, K., Baker, S. J., Cosenza, S. C., Basu, I., Gupta, Y. K., Reddy, M. V., Ueno, L., Hart, J. R., et al. (2016). A Small Molecule RAS-Mimetic Disrupts RAS Association with Effector Proteins to Block Signaling. Cell 165, 643-655.
31. di Magliano, M. P., and Logsdon, C. D. Roles for KRAS in pancreatic tumor development and progression. Gastroenterology 144, 1220-1229.
32. Fleming, J. B Shen, G. L., Holloway, S. E., Davis, M., and Brekken, R. A. (2005). Molecular consequences of silencing mutant K-ras in pancreatic cancer cells: justification for K-ras-directed therapy. Mol Cancer Res 3, 413-423.
33. Galanty, Y. Belotserkovskaya, R., Coates, J., Polo, S Miller, K. M., and Jackson, S. P. (2009). Mammalian SUMO E3-ligases PIAS1 and PIAS4 promote responses to DNA double-strand breaks. Nature 462, 935-939.
34. Kaikkonen, S., Jaaskelainen, T., Karvonen, U., Rytinki, M. M., Makkonen, H., Gioeli, D., Paschal, B. M., and Palvimo J. J. (2009). SUMO-specific protease 1 (SENP1) reverses the hormoneaugmented SUMOylation of androgen receptor and modulates gene responses in prostate cancer cells. Mol Endocrinol 23, 292-307.
35. Kim, Y. S., Keyser, S. G., and Schneekloth, J. S., Jr. (2014). Synthesis of 2′,3′,4′-trihydroxyflavone (2-D08), an inhibitor of protein sumoylation. Bioorg Med Chem Lett 24, 1094-1097.
36. Landre, V., Rotblat, B., Melino, S., Bernassola, F., and Melina, G. (2014). Screening for E3-ubiquitin ligase inhibitors: challenges and opportunities. Oncotarget 5, 7988-8013.
37. Niewerth, D., Dingjan, I., Cloos, J., Jansen, G., and Kaspers, G. (2013). Proteasome inhibitors in acute leukemia. Expert Rev Anticancer Ther 13, 327-337.
38. Rodriguez-Viciana, P., Warne, P. H., Khwaja, A., Marte, B. M., Pappin, D., Das, P., Waterfield, M. D., Ridley, A., and Downward, J. (1997). Role of phosphoinositide 3-OH kinase in cell transformation and control of the actin cytoskeleton by Ras. Cell 89, 457-467.
39. Shen, L., Kim, S. H., and Chen, C. Y. (2012). Sensitization of human pancreatic cancer cells harboring mutated K-ras to apoptosis. PLoS One 7, e40435.
40. Shih, J. Y., and Yang, P. C. (2011). The EMT regulator slug and lung carcinogenesis. Carcinogenesis 32, 1299-1304.
41. Tsai, F. D., Lopes, M. S., Zhou, M., Court, H., Ponce, O., Fiordalisi, J. J., Gierut, J. J., Cox, A. D., Haigis, K. M., and Philips, M. R. K-Ras4A splice variant is widely expressed in cancer and uses a hybrid membrane-targeting motif. Proc Natl Acad Sci U S A 112, 779-784.
42. Sambrook, J., Fritsch, E. F., and Maniatis, T. Molecular cloning: A laboratory manual. Second edition. Cold Spring Harbor, New York: Cold Spring Harbor Laboratory Press. (1989).
43. Stayer, L., Biochemistry, 4th ed., WH Freeman and Co.: 1995.

What is claimed is:

1. A method for modulating activity of a Ras protein in a cell, the method comprising:
contacting the cell with an agent, wherein the agent interferes with sumoylation of the Ras protein at amino acid residue 42 by a small ubiquitin-like modifier (SUMO) protein.

2. The method of claim 1, wherein the Ras protein comprises a point mutation at amino acid residue 12, 13, 17, 61 and/or 119 of the Ras protein.

3. The method of claim 1, wherein the Ras protein comprises the amino acid sequence set forth in SEQ ID NO: 2 comprising the point mutation at amino acid residue 12, 13, 17, 61 and/or 119 of SEQ ID NO:2.

4. The method of claim 1, wherein the agent is an antibody or a fragment thereof that specifically binds to an epitope comprising amino acid residue 42 of the Ras protein.

5. The method of claim 1, wherein the agent is a SUMO E2 inhibitor.

6. The method of claim 5, wherein the SUMO E2 inhibitor is 2-(2,3,4-Trihydroxyphenyl)-4H-chromen-4-one (2-D08).

7. The method of claim 5, wherein the SUMO E2 inhibitor is a derivative, an analog, a variant, or a salt of 2-D08.

8. The method of claim 1, wherein the agent is a SUMO E3 inhibitor.

9. The method of claim 8, wherein the SUMO E3 inhibitor is an inhibitor of PIASγ.

10. The method of claim 1, wherein the agent is an activator of a sentrin-specific protease (SENP).

11. The method of claim 1, wherein the SUMO protein is SUMO-3.

12. The method of claim 1, wherein cell proliferation and/or migration is decreased.

13. A method for decreasing proliferation and/or migration of a cancer cell comprising a Ras protein, the method comprising: contacting the cell with an agent, wherein the agent interferes with sumoylation of the Ras protein at amino acid residue 42 by a SUMO protein.

14. The method of claim 13, wherein the Ras protein comprises a point mutation at amino acid residue 12, 13, 17, 61 and/or 119 of the Ras protein.

15. The method of claim 13, wherein the Ras protein comprises the amino acid sequence set forth in SEQ ID NO: 2 comprising a point mutation at amino acid residue 12, 13, 17, 61 and/or 119 of SEQ ID NO:2.

16. The method of claim 13, wherein the agent is a SUMO E2 inhibitor.

17. The method of claim 16, wherein the SUMO E2 inhibitor is 2-(2,3,4-Trihydroxyphenyl)-4H-chromen-4-one (2-D08).

18. The method of claim 16, wherein the SUMO E2 inhibitor is a derivative, an analog, a variant, or a salt of 2-D08.

19. The method of claim 13, wherein the agent is a SUMO E3 inhibitor.

20. The method of claim 19, wherein the SUMO E3 inhibitor is an inhibitor of PIASγ.

21. The method of claim 13, wherein the aunt is an activator of a sentrin-specific protease (SENP).

22. The method of claim 13, wherein the cancer cell is a lung cancer cell or a pancreatic cancer cell.

23. A method for decreasing sumoylation of a Ras protein by a small ubiquitin-like modifier (SUMO) protein, the method comprising: introducing a first point mutation at amino acid residue 42 of the Ras protein to form a modified Ras protein.

24. The method of claim 23, wherein the Ras protein comprises a second point mutation at amino acid residue 12, 13, 17, 61 or 119 of the Ras protein.

25. The method of claim 23, wherein the Ras protein comprises the amino acid sequence set forth in SEQ ID NO: 2 further comprising the first point mutation at amino acid residue 42 of SEQ ID NO: 2.

26. A modified Ras protein comprising the amino acid sequence set forth in SEQ ID NO: 2 comprising a point mutation at amino acid residue 42 of SEQ ID NO:2.

27. A modified Ras protein comprising the amino acid sequence set forth in SEQ ID NO: 2 comprising the following mutations: (i) a first point mutation at amino acid residue 42 of SEQ ID NO:2 substituted with an amino acid other than Lysine; and (ii) a second point mutation at amino acid residue 12 of SEQ ID NO:2 substituted with a valine or an amino acid other than glycine, at amino acid residue 13 of SEQ ID NO:2 substituted with an amino acid other than glycine, at amino acid residue 17 of SEQ ID NO:2 substituted with asparagine or an amino acid other than serine, at amino acid residue 61 of SEQ ID NO:2 substituted with an amino acid other than glutamine, and/or at amino acid residue 119 of SEQ ID NO:2 substituted with asparagine or an amino acid other than aspartic acid.

28. A nucleic acid encoding the modified Ras protein of claim 27.

29. A recombinant vector comprising the nucleic acid of claim 28.

30. A cell comprising the nucleic acid of claim 28.


SEQUENCE LISTING (12 sequences)

SEQ ID NO: 1 - DNA, 570 bp, Human
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 2 - protein, 189 aa, Human
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - DNA, 570 bp, Human
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 4 - protein, 189 aa, Human
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 5 - DNA, 570 bp, Human
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaa

SEQ ID NO: 6 - protein, 189 aa, Human [sequence omitted]

SEQ ID NO: 7 - DNA, 567 bp, Human
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 8 - protein, 188 aa, Human [sequence omitted]

SEQ ID NO: 9 - protein, 101 aa, Human
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 10 - protein, 95 aa, Human
MADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 11 - protein, 103 aa, Human
MSEEKPKEGVKTENDHINLKVAGQDGSVVQFKIKRHTPLSKLMKAYCERQGLSMRQIRFRFDGQPINETDTPAQLEMEDEDTIDVFQQQTGGVPESSLAGHSF

SEQ ID NO: 12 - protein, 95 aa, Human
MANEKPTEEVKTENNNHINLKVAGQDGSVVQFKIKRQTPLSKLMKAYCEPRGLSVKQIRFRFGGQPISGTDKPAQLEMEDEDTIDVFQQPTGGVY